SEQUENCE LISTING (78 sequences)

SEQ ID NO: 1 - protein, 217 aa, Artificial Sequence
  Synthetic Sequence_Fc domain linkers and joining linkers
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Sequence_Fc domain linkers and joining linkers
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Sequence_Fc domain linkers and joining linkers
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Sequence_Fc domain linkers and joining linkers
SKYGPPCPSCP

SEQ ID NO: 5 - protein, 11 aa, Artificial Sequence
  Synthetic Sequence_Fc domain linkers and joining linkers
SKYGPPCPPCP

SEQ ID NO: 6 - protein, 6 aa, Artificial Sequence
  Synthetic Sequence_Fc domain linkers and joining linkers
SKYGPP

SEQ ID NO: 7 - protein, 6 aa, Artificial Sequence
  Synthetic Sequence_Fc domain linkers and joining linkers
IEGRMD

SEQ ID NO: 8 - protein, 9 aa, Artificial Sequence
  Synthetic Sequence_Fc domain linkers and joining linkers
GGGVPRDCG

SEQ ID NO: 9 - protein, 15 aa, Artificial Sequence
  Synthetic Sequence_Fc domain linkers and joining linkers
IEGRMDGGGGAGGGG

SEQ ID NO: 10 - protein, 8 aa, Artificial Sequence
  Synthetic Sequence_Fc domain linkers and joining linkers
GGGSGGGS

SEQ ID NO: 11 - protein, 12 aa, Artificial Sequence
  Synthetic Sequence_Fc domain linkers and joining linkers
GGGSGGGGSGGG

SEQ ID NO: 12 - protein, 14 aa, Artificial Sequence
  Synthetic Sequence_Fc domain linkers and joining linkers
EGKSSGSGSESKST

SEQ ID NO: 13 - protein, 4 aa, Artificial Sequence
  Synthetic Sequence_Fc domain linkers and joining linkers
GGSG

SEQ ID NO: 14 - protein, 12 aa, Artificial Sequence
  Synthetic Sequence_Fc domain linkers and joining linkers
GGSGGGSGGGSG

SEQ ID NO: 15 - protein, 15 aa, Artificial Sequence
  Synthetic Sequence_Fc domain linkers and joining linkers
EAAAKEAAAKEAAAK

SEQ ID NO: 16 - protein, 20 aa, Artificial Sequence
  Synthetic Sequence_Fc domain linkers and joining linkers
EAAAREAAAREAAAREAAAR

SEQ ID NO: 17 - protein, 17 aa, Artificial Sequence
  Synthetic Sequence_Fc domain linkers and joining linkers
GGGGSGGGGSGGGGSAS

SEQ ID NO: 18 - protein, 9 aa, Artificial Sequence
  Synthetic Sequence_Fc domain linkers and joining linkers
GGGGAGGGG

SEQ ID NO: 19 - skipped

SEQ ID NO: 20 - protein, 6 aa, Artificial Sequence
  Synthetic Sequence_Fc domain linkers and joining linkers
GSGSGS

SEQ ID NO: 21 - protein, 10 aa, Artificial Sequence
  Synthetic Sequence_Fc domain linkers and joining linkers
GSGSGSGSGS

SEQ ID NO: 22 - protein, 7 aa, Artificial Sequence
  Synthetic Sequence_Fc domain linkers and joining linkers
GGGGSAS

SEQ ID NO: 23 - protein, 20 aa, Artificial Sequence
  Synthetic Sequence_Fc domain linkers and joining linkers
APAPAPAPAPAPAPAPAPAP

SEQ ID NO: 24 - protein, 4 aa, Artificial Sequence
  Synthetic Sequence_Fc domain linkers and joining linkers
CPPC

SEQ ID NO: 25 - protein, 5 aa, Artificial Sequence
  Synthetic Sequence_Fc domain linkers and joining linkers
GGGGS

SEQ ID NO: 26 - protein, 10 aa, Artificial Sequence
  Synthetic Sequence_Fc domain linkers and joining linkers
GGGGSGGGGS

SEQ ID NO: 27 - protein, 15 aa, Artificial Sequence
  Synthetic Sequence_Fc domain linkers and joining linkers
GGGGSGGGGSGGGGS

SEQ ID NO: 28 - protein, 20 aa, Artificial Sequence
  Synthetic Sequence_Fc domain linkers and joining linkers
GGGGSGGGGSGGGGSGGGGS

SEQ ID NO: 29 - protein, 25 aa, Artificial Sequence
  Synthetic Sequence_Fc domain linkers and joining linkers
GGGGSGGGGSGGGGSGGGGSGGGGS

SEQ ID NO: 30 - protein, 30 aa, Artificial Sequence
  Synthetic Sequence_Fc domain linkers and joining linkers
GGGGSGGGGSGGGGSGGGGSGGGGSGGGGS

SEQ ID NO: 31 - protein, 35 aa, Artificial Sequence
  Synthetic Sequence_Fc domain linkers and joining linkers
GGGGSGGGGSGGGGSGGGGSGGGGSGGGGSGGGGS

SEQ ID NO: 32 - protein, 40 aa, Artificial Sequence
  Synthetic Sequence_Fc domain linkers and joining linkers
GGGGSGGGGSGGGGSGGGGSGGGGSGGGGSGGGGSGGGGS

SEQ ID NO: 33 - protein, 16 aa, Artificial Sequence
  Synthetic Sequence_Fc domain linkers and joining linkers
GGSGGSGGGGSGGGGS

SEQ ID NO: 34 - protein, 8 aa, Artificial Sequence
  Synthetic Sequence_Fc domain linkers and joining linkers
GGGGGGGG

SEQ ID NO: 35 - protein, 6 aa, Artificial Sequence
  Synthetic Sequence_Fc domain linkers and joining linkers
GGGGGG

SEQ ID NO: 36 - protein, 5 aa, Artificial Sequence
  Synthetic Sequence_Fc domain linkers and joining linkers
EAAAK

SEQ ID NO: 37 - protein, 10 aa, Artificial Sequence
  Synthetic Sequence_Fc domain linkers and joining linkers
EAAAKEAAAK

SEQ ID NO: 38 - protein, 15 aa, Artificial Sequence
  Synthetic Sequence_Fc domain linkers and joining linkers
EAAAKEAAAKEAAAK

SEQ ID NO: 39 - protein, 12 aa, Artificial Sequence
  Synthetic Sequence_Fc domain linkers and joining linkers
AEAAAKEAAAKA

SEQ ID NO: 40 - protein, 17 aa, Artificial Sequence
  Synthetic Sequence_Fc domain linkers and joining linkers
AEAAAKEAAAKEAAAKA

SEQ ID NO: 41 - protein, 22 aa, Artificial Sequence
  Synthetic Sequence_Fc domain linkers and joining linkers
AEAAAKEAAAKEAAAKEAAAKA

SEQ ID NO: 42 - protein, 27 aa, Artificial Sequence
  Synthetic Sequence_Fc domain linkers and joining linkers
AEAAAKEAAAKEAAAKEAAAKEAAAKA

SEQ ID NO: 43 - protein, 46 aa, Artificial Sequence
  Synthetic Sequence_Fc domain linkers and joining linkers
AEAAAKEAAAKEAAAKEAAAKALEAEAAAKEAAAKEAAAKEAAAKA

SEQ ID NO: 44 - protein, 5 aa, Artificial Sequence
  Synthetic Sequence_Fc domain linkers and joining linkers
PAPAP

SEQ ID NO: 45 - protein, 18 aa, Artificial Sequence
  Synthetic Sequence_Fc domain linkers and joining linkers
KESGSVSSEQLAQFRSLD

SEQ ID NO: 46 - protein, 12 aa, Artificial Sequence
  Synthetic Sequence_Fc domain linkers and joining linkers
GSAGSAAGSGEF

SEQ ID NO: 47 - protein, 5 aa, Artificial Sequence
  Synthetic Sequence_Fc domain linkers and joining linkers
GGGSE

SEQ ID NO: 48 - protein, 5 aa, Artificial Sequence
  Synthetic Sequence_Fc domain linkers and joining linkers
GSESG

SEQ ID NO: 49 - protein, 5 aa, Artificial Sequence
  Synthetic Sequence_Fc domain linkers and joining linkers
GSEGS

SEQ ID NO: 50 - protein, 35 aa, Artificial Sequence
  Synthetic Sequence_Fc domain linkers and joining linkers
GEGGSGEGSSGEGSSSEGGGSEGGGSEGGGSEGGS

SEQ ID NO: 51 - protein, 234 aa, Artificial Sequence
  Synthetic Sequence_Modular Linker
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Sequence_Modular Linker
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Sequence_Modular Linker
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Sequence_Modular Linker
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Sequence_Modular Linker
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Sequence_Modular Linker
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 288 aa, Artificial Sequence
  Synthetic Sequence_wild type PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 58 - protein, 143 aa, Artificial Sequence
  Synthetic Sequence_wild type PD-1
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 59 - protein, 143 aa, Artificial Sequence
  Synthetic Sequence_wild type PD-1
LDSPDRPWNPPTFSPALLVVTEGDNATFTCSFSNTSESFVLNWYRMSPSNQTDKLAAFPEDRCQPGQDCRFRVTQLPNGRDFHMSVVRARRNDSGTYLCGAISLAPKAQIKESLRAELRVTERRAEVPTAHPSPSPRPAGQFQ

SEQ ID NO: 60 - protein, 143 aa, Artificial Sequence
  Synthetic Sequence_wild type PD-1
LDSPDRPWNPPTFSPALLVVTEGDNATFTCSFSNTSESFVLNWYRMSPSNQTDKLAAFPEDACQPGQDCRFRVTQLPNGRDFHMSVVRARRNDSGTYLCGAISLAPKAQIKESLRAELRVTERRAEVPTAHPSPSPRPAGQFQ

SEQ ID NO: 61 - protein, 143 aa, Artificial Sequence
  Synthetic Sequence_wild type PD-1
LDSPDRPWNPPTFSPALLVVTEGDNATFTCSFSNTSESFVLNWYRMSPSNQTDKLAAFPEDRCEPGQDCRFRVTQLPNGRDFHMSVVRARRNDSGTYLCGAISLAPKAQIKESLRAELRVTERRAEVPTAHPSPSPRPAGQFQ

SEQ ID NO: 62 - protein, 143 aa, Artificial Sequence
  Synthetic Sequence_wild type PD-1
LDSPDRPWNPPTFSPALLVVTEGDNATFTCSFSNTSESFVLNWYRMSPSNQTDKLAAFPEDACEPGQDCRFRVTQLPNGRDFHMSVVRARRNDSGTYLCGAISLAPKAQIKESLRAELRVTERRAEVPTAHPSPSPRPAGQFQ

SEQ ID NO: 63 - skipped

SEQ ID NO: 64 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 65 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 66 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 67 - protein, 172 aa, Homo sapiens
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 68 - protein, 215 aa, Homo sapiens
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 69 - protein, 128 aa, Homo sapiens
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 70 - protein, 195 aa, Homo sapiens
SQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 71 - protein, 182 aa, Homo sapiens
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 72 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 73 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 74 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 75 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 76 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 77 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 78 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

(19) United States
(12) Patent Application Publication
(10) Pub. No.: US 2024/0141014 A1
(43) Pub. Date: May 2, 2024

(54) MUTANT PD-1 EXTRACELLULAR DOMAINS

(71) Applicant: Shattuck Labs, Inc., Austin, TX (US)

(72) Inventors: Taylor SCHREIBER, Austin, TX (US); Suresh DE SILVA, Austin, TX (US); George FROMM, Austin, TX (US)

(21) Appl. No.: 18/279,131

(22) PCT Filed: Mar. 3, 2022
(86) PCT No.: PCT/US22/18710

§ 371 Date: Aug. 28, 2023

Related U.S. Application Data
(60) Provisional application No. 63/197,782, filed on Jun. 7, 2021.
(60) Provisional application No. 63/156,007, filed on Mar. 3, 2021.

(51) Int. Cl. C07K 14/705; C12N 15/63

(52) U.S. Cl. CPC C07K 14/70521; C07K 14/70575; C12N 15/63; C07K 2319/30

(57) ABSTRACT

The current disclosure relates to, inter alia, mutant derivatives of PD-1 protein. The current disclosure also relates to compositions and methods that find use in the treatment of diseases, such as immunotherapies for cancer and autoimmunity.

### PRIORITY

This application claims the benefit of, and priority to, U.S. Provisional Application Nos. 63/197,782, filed Jun. 7, 2021; and 63/156,007, filed Mar. 3, 2021, the contents of each which are hereby incorporated by reference in their entirety.

### TECHNICAL FIELD

The current disclosure relates to mutant derivatives of PD-1 protein. These mutant derivatives find use in the treatment of diseases, such as immunotherapies for cancer and autoimmunity.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SHK-043PC_116981-5043_ST25; date created: Mar. 2, 2022; file size: 67,321 bytes).

### BACKGROUND

PD-1 is an inhibitory T-cell surface receptor that suppresses the T-cell activation. Its receptors are PD-L1 and PD-L2, which are often overexpressed in tumor cells, while PD-1 is highly expressed on T cells in patient tumors. Upon binding to PD-L1 or PD-L2, the PD-1 receptor blocks signaling in T cells, and thereby suppressing anti-tumor immune response. Anti-PD-1 antibodies, including pembrolizumab, nivolumab, and cemiplimab have approved for clinical use. It has been shown that the average serum half-life of these antibodies is 2-4 weeks. Centanni et al., Clinical Pharmacokinetics and Pharmacodynamics of Immune Checkpoint Inhibitors, Clinical Pharmacokinetics 58: 835-857(2019). The antibodies persist in sera of patients that are administered these antibodies for months. Persistent antibodies may affect other targeted therapies a patient is receiving. Therefore, agents that account for the residual antibodies are required.

### SUMMARY

Accordingly, in one aspect, the current disclosure provides a variant extracellular domain (ECD) of PD-1, and proteins comprising the same. In various aspects, the present invention provides for compositions and methods that are useful for cancer and antiviral immunotherapy. For example, in various embodiments, the present compositions and methods allow for a therapeutic benefit that is not reduced or eliminated by residual antibodies in sera from prior treatments. For instance, in embodiments, the present disclosure relates to variant PD-1 extracellular domains, which can evade binding by an anti-PD-1 antibody, e.g., from a prior treatment, e.g., nivolumab and/or pembrolizumab, e.g., KEYTRUDA or OPDIVO.

In one aspect, the present disclosure relates to a polypeptide comprising a variant extracellular domain (ECD) of PD-1, wherein the variant ECD comprises one or more substitutions at one or more amino acid residues corresponding to one or more of an arginine residue at the position 86 (R86), a serine residue at the position 87 (S87), and a glutamine residue at the position 88 (Q88) with respect to SEQ ID NO: 57, wherein the variant ECD and/or the polypeptide has less affinity to pembrolizumab and/or nivolumab compared to a wild type PD-1 ECD having an amino acid sequence that is identical to the amino acid sequence of SEQ ID NO: 58.

In one aspect, the present disclosure relates to a chimeric protein comprising: (a) a variant extracellular domain (ECD) of PD-1, wherein the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57; and (b) a carrier protein, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58. In embodiments, the carrier protein is selected from albumin, transferrin, an Fc, or elastin-like protein, or a variant thereof. In embodiments, the Fc domain is selected from an IgG Fc domain, an IgA Fc domain, an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the Fc domain comprises hinge-CH2-CH3 Fc domain derived from IgG4. In embodiments, the Fc domain the hinge-CH2-CH3 Fc domain is derived from human IgG4. In embodiments, the Fc domain comprises an amino acid sequence that is at least 95% identical to the amino acid sequence of SEQ ID NO: 1, SEQ ID NO: 2, or SEQ ID NO: 3. In embodiments, the chimeric protein further comprises one or more joining linkers, such joining linkers independently selected from SEQ ID NOs: 4-50.

In one aspect, the present disclosure relates to a chimeric protein comprising (a) a first domain comprising a variant extracellular domain (ECD) of PD-1, wherein the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57, (b) a second domain comprising an extracellular domain of a transmembrane protein, and (c) a linker, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58. In embodiments, the linker comprises a polypeptide selected from a flexible amino acid sequence, an IgG hinge region, and an antibody sequence. In embodiments, the linker comprises at least one cysteine residue capable of forming a disulfide bond. In embodiments, the linker comprises an Fc domain. In embodiments, the Fc domain is selected from an IgG Fc domain, an IgA Fc domain, an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the Fc domain comprises hinge-CH2-CH3 Fc domain derived from IgG4. In embodiments, the Fc domain the hinge-CH2-CH3 Fc domain is derived from human IgG4. In embodiments, the Fc domain comprises an amino acid sequence that is at least 95% identical to the amino acid sequence of SEQ ID NO: 1, SEQ ID NO: 2, or SEQ ID NO: 3. In embodiments, the Fc domain comprises hinge-CH2-CH3 Fc domain derived from IgG1. In embodiments, the Fc domain the hinge-CH2-CH3 Fc domain is derived from human IgG1. In embodiments, the chimeric protein further comprises one or more joining linkers, such joining linkers independently selected from SEQ ID NOs: 4-50. In embodiments, the linker comprises two or more joining linkers each joining linker independently selected from SEQ ID NOs: 4-50; wherein one joining linker is N terminal to the hinge-CH2-CH3 Fc domain and another joining linker is C terminal to the hinge-CH2-CH3 Fc domain. In embodiments, the transmembrane protein is a Type II transmembrane protein. In embodiments, the Type II transmembrane protein is selected from 4-1BBL, OX40L, CD70, CD30L, CD40L, GITRL, TL1A, and LIGHT.

In embodiments, the variant ECD comprises an amino acid substitution at an amino acid corresponding to the arginine residue at the position 86 (R86) with respect to SEQ ID NO: 57. In embodiments, the variant ECD comprises an amino acid substitution at an amino acid corresponding to the serine residue at the position 87 (S87) with respect to SEQ ID NO: 57. In embodiments, the variant ECD comprises an amino acid substitution at an amino acid corresponding to the glutamine residue at the position 88 (Q88) with respect to SEQ ID NO: 57.

In embodiments, the variant ECD comprises an amino acid substitution at an amino acid corresponding to R86 and S87 with respect to SEQ ID NO: 57. In embodiments, the variant ECD comprises an amino acid substitution at an amino acid corresponding to S87 and Q88 with respect to SEQ ID NO: 57. In embodiments, the variant ECD comprises an amino acid substitution at an amino acid corresponding to R86 and Q88 with respect to SEQ ID NO: 57. In embodiments, the variant ECD comprises an amino acid substitution at an amino acid corresponding to R86, S87, and Q88 with respect to SEQ ID NO: 57.

In embodiments, S87 with respect to SEQ ID NO: 57 is replaced with an hydrophilic, polar and neutral of charge amino acid residue selected from asparagine (N), glutamine (Q), threonine (T), proline (P), and cysteine (C). In embodiments, S87 with respect to SEQ ID NO: 57 is replaced with cysteine (C). In embodiments, the variant ECD comprises a S87C substitution with respect to SEQ ID NO: 57.

Additionally or alternatively, in embodiments, the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the hydrophobic, aliphatic amino acid residue is alanine (A). In embodiments, the variant ECD comprises a R86A substitution with respect to SEQ ID NO: 57.

Additionally or alternatively, in embodiments, the Q88 with respect to SEQ ID NO: 57 is replaced with a polar and negatively charged hydrophilic amino acid residue. In embodiments, the polar and negatively charged hydrophilic amino acid residue is glutamic acid (E). In embodiments, the variant ECD comprises a Q88E substitution with respect to SEQ ID NO: 57.

In one aspect, the present disclosure relates to a recombinant protein comprising variant extracellular domain (ECD) of PD-1 comprising an amino acid sequence that is 95% identical to the amino acids of (i) SEQ ID NO: 59, or (ii) SEQ ID NO: 60, (iii) SEQ ID NO: 61, or (iv) SEQ ID NO: 62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57.

In one aspect, the present disclosure relates to a chimeric protein comprising: (a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the isolated mutant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57; and (b) a carrier protein selected from selected from albumin, transferrin, an Fc, or elastin-like protein, or a variant thereof.

In one aspect, the present disclosure relates to a chimeric protein comprising (a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the isolated mutant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57; (b) a second domain comprising an extracellular domain of a Type II transmembrane protein selected from 4-1BBL, OX40L, CD70, CD30L, CD40L, GITRL, TL1A, and LIGHT, and (c) a linker. In embodiments, the chimeric protein comprises a general structure of: N terminus-(a)-(c)-(b)-C terminus, wherein: (c) is the linker, and (b) is the second domain comprising an extracellular domain of Type II transmembrane protein.

In embodiments, the variant ECD, recombinant protein, the chimeric protein, and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the variant ECD, recombinant protein, the chimeric protein, and/or the chimeric protein has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2.

In one aspect, the present disclosure relates to a nucleic acid encoding the recombinant protein of any of the embodiments disclosed herein, chimeric protein of any of the embodiments disclosed herein, or the chimeric protein of any of the embodiments disclosed herein. In embodiments, the nucleic acid is an mRNA. In embodiments, the nucleic acid is a DNA.

In one aspect, the present disclosure relates to an expression vector, comprising a nucleic acid encoding the chimeric protein of any of the embodiments disclosed herein.

In one aspect, the present disclosure relates to a host cell, comprising the expression vector of any of the embodiments disclosed herein, mRNA of any of the embodiments disclosed herein, or DNA of any of the embodiments disclosed herein.

In one aspect, the present disclosure relates to a pharmaceutical composition, comprising a therapeutically effective amount of the recombinant protein of any of the embodiments disclosed herein, chimeric protein of any of the embodiments disclosed herein, the chimeric protein of any of the embodiments disclosed herein, or the nucleic acid of any of the embodiments disclosed herein, or the expression vector of any of the embodiments disclosed herein, or the host cell of any of the embodiments disclosed herein.

In one aspect, the present disclosure relates to a method of treating cancer or an inflammatory disease, comprising administering an effective amount of a pharmaceutical composition of any of the embodiments disclosed herein to a subject in need thereof.

### BRIEF DESCRIPTION OF THE DRAWINGS

FIG. 1A to FIG. 1C show the binding of the PD-1-Fc-4-1BBL chimeric protein or its mutant derivatives to PD-L1, nivolumab and pembrolizumab as determined using the Meso Scale Discovery (MSD) ELISA assays. The mutant derivatives were PD-1(R86A)-Fc-4-1BBL, PD-1(Q88E)-Fc-4-1BBL, and PD-1(R86A-Q88E)-Fc-4-1BBL. FIG. 1A shows the binding of PD-1-Fc-4-1BBL chimeric protein or its mutant derivatives to nivolumab. FIG. 1B shows the binding of PD-1-Fc-4-1BBL chimeric protein or its mutant derivatives to PD-L1. FIG. 1C shows the locations of R86, S87, and Q88 on the surface of PD-1.

FIG. 2A to FIG. 2F show the binding of the PD-1-Fc fusion protein and its mutant derivatives to pembrolizumab and PD-L1 as measured by Bio-layer Interferometry using the Octet system (ForteBio). FIG. 2A shows the binding curves of the PD-1-Fc fusion protein to pembrolizumab at increasing concentrations of the PD-1-Fc fusion protein. FIG. 2B shows the binding curves of the mutant PD-1-Fc fusion proteins, designated as D1, E1, C3, F3, and G3. H3 was a commercially available PD-1-Fc, which showed no activity in this assay. FIG. 2C shows the kinetic parameters (kₒₙ, k_{dis}, K_{D} and R²) of the binding of the PD-1-Fc fusion protein or the mutants thereof to pembrolizumab. FIG. 2D shows the binding curves of the PD-1-Fc fusion protein to PD-L1 at increasing concentrations of the PD-1-Fc fusion protein. FIG. 2E shows the binding curves of the mutant PD-1-Fc fusion proteins D1, E1, C3, F3, and G3. FIG. 2F shows the kinetic parameters (kₒₙ, k_{dis}, K_{D} and R²) for the binding of the PD-1-Fc fusion protein or the mutants thereof to PD-1.

FIG. 3A and FIG. 3D show the effect of binding of single substitution mutants at S87, and double or triple substitutions at S87, and R86 and/or Q88 of PD-1 on the binding to pembrolizumab, PD-L1, or nivolumab as determined using the Meso Scale Discovery (MSD) ELISA assays. FIG. 3A shows the binding curves of the PD-1-Fc fusion protein or the indicated mutants thereof to pembrolizumab. FIG. 3B shows the binding curves of the PD-1-Fc fusion protein or the indicated mutants thereof to PD-L1. FIG. 3C shows the binding curves of the PD-1-Fc fusion protein or the indicated mutants thereof to nivolumab. FIG. 3D shows the summary of the binding of the PD-1-Fc fusion protein or the mutants thereof to PD-1.

FIG. 4A to FIG. 4C show the binding of the PD-1-Fc-OX40L chimeric fusion protein and its mutant derivatives to pembrolizumab (FIG. 4A), PD-L1 (FIG. 4B), or nivolumab (FIG. 4C) as measured by Meso Scale Discovery (MSD) ELISA assays. Pembrolizumab, PD-L1, or nivolumab were coated on plates. Increasing amounts of the PD-1-Fc-OX40L chimeric fusion protein, or its mutant derivatives, were added to the plates for capture by the plate-bound pembrolizumab, PD-L1, or nivolumab. Binding was detected using an anti-Fc antibody.

FIG. 5 demonstrates that pembolizumab does not inhibit the binding of the mutant derivatives of the PD-1-Fc-OX40L chimeric fusion protein disclosed herein to cells expressing PD-1 on their surface. The PD-1-Fc-OX40L chimeric fusion protein or mutants thereof were pre-incubated with either buffer only, nivolumab, or pembrolizumab. The mixture was added to CHO-K1 cells expressing human PD-L1 (the CHO-K1/hPD-L1 cells). Binding of the PD-1-Fc-OX40L chimeric fusion protein or mutants thereof to the CHO-K1/hPD-L1 cells was detected using an anti-Fc antibody and measured by flow cytometry.

### DETAILED DESCRIPTION

The present disclosure is based, in part on the discovery that certain variant extracellular domains (ECDs) disclosed herein show lower binding affinity to the anti-PD-1 antibody pembrolizumab, while retaining the binding to the PD-1 ligand PD-L1. In embodiments, the variant ECDs of PD-1 disclosed herein are useful for accounting for the anti-PD-1 antibodies that may be present in patients. The variant ECDs of PD-1 disclosed herein are useful, without limitation, for avoiding interference in the therapeutic effect of agents that are capable of binding anti-PD-1 antibodies (without limitation, e.g., the therapeutic agents that include ECD of PD-1), by anti-PD-1 antibodies (without limitation, e.g., pembrolizumab) present in sera of patients. In embodiments, the anti-PD-1 antibodies in sera of patients may be from prior treatments. In embodiments, the anti-PD-1 antibodies in sera of patients may be from combination treatment. For example, variant ECDs of PD-1 disclosed herein are useful for treating patients that have or expected to have in their sera anti-PD-1 antibodies (without limitation, e.g., pembrolizumab)—including residual anti-PD-1 antibodies. Accordingly, in various embodiments, the present compositions and methods allow for a therapeutic benefit that is not reduced or eliminated by residual antibodies in sera from prior treatments and/or a combination treatment with anti-PD-1 antibodies. In embodiments, the variant PD-1 extracellular domains can evade binding by an anti-PD-1 antibody, e.g., from a prior treatment and/or from a combination treatment, e.g., nivolumab and/or pembrolizumab, e.g., KEYTRUDA and/or OPDIVO.

Accordingly, in one aspect, the present disclosure relates to a variant extracellular domain (ECD) of PD-1, wherein the variant ECD has less affinity to pembrolizumab and/or nivolumab compared to a wild type PD-1 ECD having an amino acid sequence that is identical to the amino acid sequence of SEQ ID NO: 58.

### The Variant Extracellular Domains (ECDs) of PD-1

Programmed cell death protein 1, or PD-1, is a cell surface protein present on T cells and other white blood cells. It is an inhibitory receptor on antigen activated T-cells that plays a role in induction and maintenance of immune tolerance to self. It delivers inhibitory signals upon binding to ligands CD274/PD-L1 and CD273/PD-L2. Following T-cell receptor (TCR) engagement, PD-1 associates with CD3-TCR in the immunological synapse and inhibits T-cell activation. For example, following ligand-binding, PD-1 is phosphorylated within the ITSM motif, leading to the recruitment of the protein tyrosine phosphatase SHP-2 that mediates dephosphorylation of key TCR proximal signaling molecules.

The PD-1-mediated inhibitory pathway is exploited by tumors to attenuate anti-tumor immunity and escape destruction by the immune system, thereby facilitating tumor survival. The PD-1 ligands, PD-L1 and PD-L2, can be expressed by tumor cells as well as other immune cells in the tumor microenvironment. When PD-L1 binds to PD-1, the resulting PD-1 signaling limits the capacity of T cells to kill tumor cells. PD-L1 and PD-L2 can be expressed by tumor cells (and also other immune cells in the tumor microenvironment) and bind to PD-1 receptor expressed by tumor infiltrating lymphocytes. When this occurs, PD-1 signaling in lymphocytes, including T cells, limits the capacity of those T cells to kill tumor cells.

The interaction with CD274/PD-L1 inhibits cytotoxic T lymphocytes (CTLs) effector function. Inhibition of the PD-1 receptor on lymphocytes by PD-1 (or PD-L1) blocking antibodies (including and pembrolizumab (KEYTRUDA) and nivolumab (OPDIVO)) limits PD-L1 binding to PD-1, and thus maintains the baseline capacity of T cells to kill tumor cells. Anti-PD-1 antibodies disrupt binding of PD-1 to PD-L1 to restore baseline tumor cell-killing activity of T cells. Pembrolizumab is the first line treatment for PD-L1 positive advanced or metastatic non-small cell lung cancer, unresectable or metastatic microsatellite instability-high (MSI-H) or mismatch repair deficient (dMMR) colorectal cancer. Nivolumab is used in combination with the anti-CTLA antibody ipilimumab (YERVOY) as the first line treatment for metastatic non-small cell lung cancer, and malignant pleural mesothelioma. While anti-PD-1/PD-L1 antibodies have achieved significant clinical and commercial success, a majority of patients with cancer do not benefit from this class of therapy, as evidenced by a response rate of 35% or less in patients with melanoma, NSCLC, bladder cancer, HNSCC, and other cancers. A limitation of anti-PD-1/PD-L1 antibodies is their inability to provide a signal that directly amplifies the ability of T cells to kill tumor cells. Achieving this enhanced tumor-killing effect necessitates the introduction of a distinct mechanism to complement checkpoint blockade. In addition, the anti-PD-1 antibodies persist in the serum for a long time because of their long half-lives, neutralizing some of the PD-1-based therapeutics that may be subsequently administered.

Accordingly, in embodiments, the variant extracellular domain (ECD) of PD-1 disclosed herein harbor substitutions of amino acid residues of ECD of PD-1 that are involved in binding to pembrolizumab and/or nivolumab. Accordingly, in one aspect, the present disclosure relates to a variant extracellular domain (ECD) of PD-1, wherein the variant ECD comprises one or more mutations (e.g., a substitution, an insertion, a deletion, or a combination thereof (e.g., a substitution of an amino acid residue, a deletion of a second amino acid residue, an insertion at a third amino acid residue, or a combination of multiple such mutations)) at one or more amino acid residues corresponding to one or more of the arginine residue at the position 86 (R86), the serine residue at the position 87 (S87), and glutamine residue at the position 88 (Q88) with respect to SEQ ID NO: 57, wherein the variant ECD has less affinity to pembrolizumab and/or nivolumab compared to a wild type PD-1 ECD having an amino acid sequence that is identical to the amino acid sequence of SEQ ID NO: 58. In embodiments, the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57, wherein the variant ECD has less affinity to pembrolizumab and/or nivolumab compared to a wild type PD-1 ECD having an amino acid sequence that is identical to the amino acid sequence of SEQ ID NO: 58.

In embodiments, the variant ECD comprises an amino acid substitution at an amino acid corresponding to R86 and S87 with respect to SEQ ID NO: 57. In embodiments, the variant ECD comprises an amino acid substitution at an amino acid corresponding to S87 and Q88 with respect to SEQ ID NO: 57. In embodiments, the variant ECD comprises an amino acid substitution at an amino acid corresponding to R86 and Q88 with respect to SEQ ID NO: 57. In embodiments, the variant ECD comprises an amino acid substitution at an amino acid corresponding to R86, S87, and Q88 with respect to SEQ ID NO: 57.

It is noted that the wild type ECD of PD-1 has an amino acid sequence of SEQ ID NO: 58. Full length PD-1 sequence, which includes signal sequence, transmembrane domain, and intracellular domain, in addition to the ECD, has an amino acid sequence of SEQ ID NO: 57. While the amino acid residues of the variant ECDs are numbered with respect to SEQ ID NO: 57, the variant ECDs disclosed herein include only ECDs, i.e. the variant ECDs do not include signal sequence, transmembrane domain, and intracellular domain.

In embodiments, the variant ECD has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the affinity to pembrolizumab and/or nivolumab is assessed based on the measurement of K_{D} value as shown in Example 3. In embodiments, the affinity to pembrolizumab and/or nivolumab may be assessed using as assay including, but not limited to, ELISA, Surface plasmon resistance (SPR), and bio-layer interferometry. In embodiments, the variant ECD has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2. In embodiments, the affinity to PD-L1 and/or PD-L2 is assessed based on the measurement of K_{D} value as shown in Example 3. In embodiments, the affinity to PD-L1 and/or PD-L2 may be assessed using as assay including, but not limited to, ELISA, Surface plasmon resistance (SPR), and bio-layer interferometry.

In embodiments, the variant ECD comprises one or more mutations (e.g. a substitution of an amino acid residue, a deletion of a second amino acid residue, an insertion at a third amino acid residue, or a combination of multiple such mutations) at one or more amino acid residues corresponding to one or more of arginine residue at the position 86 (R86), serine residue at the position 87 (S87), and glutamine residue at the position 88 (Q88) with respect to SEQ ID NO: 57. In embodiments, the variant ECD comprises one or more mutations (e.g. a combination of substitutions, insertion and/or deletions) at one or more amino acid residues corresponding to one or more of the arginine residue at the position 86 (R86), the serine residue at the position 87 (S87), and glutamine residue at the position 88 (Q88) with respect to SEQ ID NO: 57. In embodiments, the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57.

In embodiments, S87 with respect to SEQ ID NO: 57 is replaced with an amino acid residue that is aromatic, aliphatic, hydrophobic, polar, hydrophilic, neutral of charge, negatively charged, positively charged or combination thereof. In embodiments, S87 with respect to SEQ ID NO: 57 is replaced with an amino acid residue that is an hydrophilic, polar and positively charged residue is selected from lysine (K) and arginine (R). In embodiments, S87 with respect to SEQ ID NO: 57 is replaced with an amino acid residue that is an aromatic, polar and positively charged hydrophilic residue, wherein the aromatic, polar and positively charged hydrophilic residue is histidine (H). In embodiments, S87 with respect to SEQ ID NO: 57 is replaced with a hydrophilic is a polar and negatively charged hydrophilic amino acid residue selected from aspartate (D) and glutamate (E). In embodiments, S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, S87 with respect to SEQ ID NO: 57 is replaced with an hydrophilic, polar and neutral of charge amino acid residue selected from asparagine (N), glutamine (Q), threonine (T), proline (P), and cysteine (C). In embodiments, S87 with respect to SEQ ID NO: 57 is replaced with cysteine (C). In embodiments, the variant ECD comprises a S87C substitution with respect to SEQ ID NO: 57.

In embodiments, the R86 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the R86 with respect to SEQ ID NO: 57 is replaced with an hydrophilic, polar and positively charged residue, which is lysine. In embodiments, the R86 with respect to SEQ ID NO: 57 is replaced with an aromatic, polar and positively charged hydrophilic residue, which is histidine (H). In embodiments, the R86 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the R86 with respect to SEQ ID NO: 57 is replaced with an hydrophilic is a polar and negatively charged hydrophilic amino acid residue selected from aspartate (D) and glutamate (E). In embodiments, the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the hydrophobic, aliphatic amino acid residue is alanine (A). In embodiments, the variant ECD comprises a R86A substitution with respect to SEQ ID NO: 57. In embodiments, the variant ECD comprises a R85A, S87C double substitution.

In embodiments, the Q88 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the Q88 with respect to SEQ ID NO: 57 is replaced with an hydrophilic, polar and positively charged residue selected from arginine and lysine. In embodiments, the Q88 with respect to SEQ ID NO: 57 is replaced with an aromatic, polar and positively charged hydrophilic residue, which is histidine (H). In embodiments, the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the Q88 with respect to SEQ ID NO: 57 is replaced with an hydrophilic is a polar and negatively charged hydrophilic amino acid residue selected from aspartate (D) and glutamate (E). In embodiments, the polar and negatively charged hydrophilic amino acid residue is glutamate (E). In embodiments, the variant ECD comprises a Q88E substitution with respect to SEQ ID NO: 57. In embodiments, the variant ECD comprises a S87C, Q88E double substitution. In embodiments, the variant ECD comprises a R85A, S87C, Q88E triple substitution.

In embodiments, wild type PD-1 has the following sequence (signal sequence is shown by an underline; extracellular domains (ECD) is shown in a boldface font, transmembrane domain is shown in an italics font, and intracellular domain is shown in an underlined, italics font):

(SEQ ID NO: 57)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

In embodiments, the extracellular domain (ECD) of wild type PD-1 has the following sequence (amino acids R86, S87, and Q88 are shown in a boldface font)

(SEQ ID NO: 58)
LDSPDRPWNPPTFSPALLVVTEGDNATFTCSFSNTSESFVLNWYRMSPSN
QTDKLAAFPEDRSQPGQDCRFRVTQLPNGRDFHMSVVRARRNDSGTYLCG
AISLAPKAQIKESLRAELRVTERRAEVPTAHPSPSPRPAGQFQ.

In embodiments, the variant extracellular domain (ECD) of PD-1 S87C has the following sequence (amino acid substitution is shown in an underlined-boldface font)

(SEQ ID NO: 59)
LDSPDRPWNPPTFSPALLVVTEGDNATFTCSFSNTSESFVLNWYRMSPSN
QTDKLAAFPEDRCQPGQDCRFRVTQLPNGRDFHMSVVRARRNDSGTYLCG
AISLAPKAQIKESLRAELRVTERRAEVPTAHPSPSPRPAGQFQ.

In embodiments, the variant extracellular domain (ECD) of PD-1 R86A, S87C has the following sequence (amino acid substitutions are shown in an underlined-boldface font)

(SEQ ID NO: 60)
LDSPDRPWNPPTFSPALLVVTEGDNATFTCSFSNTSESFVLNWYRMSPSN
QTDKLAAFPEDACQPGQDCRFRVTQLPNGRDFHMSVVRARRNDSGTYLCG
AISLAPKAQIKESLRAELRVTERRAEVPTAHPSPSPRPAGQFQ.

In embodiments, the variant extracellular domain (ECD) of PD-1 S87C, Q88E has the following sequence (amino acid substitutions are shown in an underlined-boldface font)

(SEQ ID NO: 61)
LDSPDRPWNPPTFSPALLVVTEGDNATFTCSFSNTSESFVLNWYRMSPSN
QTDKLAAFPEDRCEPGQDCRFRVTQLPNGRDFHMSVVRARRNDSGTYLCG
AISLAPKAQIKESLRAELRVTERRAEVPTAHPSPSPRPAGQFQ.

In embodiments, the variant extracellular domain (ECD) of PD-1 R86A, S87C, Q88E has the following sequence (amino acid substitutions are shown in an underlined-boldface font)

(SEQ ID NO: 62)
LDSPDRPWNPPTFSPALLVVTEGDNATFTCSFSNTSESFVLNWYRMSPSN
QTDKLAAFPEDACEPGQDCRFRVTQLPNGRDFHMSVVRARRNDSGTYLCG
AISLAPKAQIKESLRAELRVTERRAEVPTAHPSPSPRPAGQFQ.

It is noted that the wild type ECD of PD-1 has an amino acid sequence of SEQ ID NO: 58. Full length PD-1 sequence, which includes signal sequence, transmembrane domain, and intracellular domain, in addition to the ECD, has an amino acid sequence of SEQ ID NO: 57. While the amino acid residues of the variant ECDs are numbered with respect to SEQ ID NO: 57, the variant ECDs disclosed herein include only ECDs, i.e. the variant ECDs do not include signal sequence, transmembrane domain, and intracellular domain.

In embodiments, the variant ECD comprises an amino acid sequence that is at least 70%, or 75%, or 80%, or 85%, or 90% identical to the amino acid sequence selected from SEQ ID NOs: 58-62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57. In embodiments, the variant ECD comprises an amino acid selected from SEQ ID NOs: 59-62. In embodiments, the variant ECD comprises an amino acid sequence that is at least 95%, or 96%, or 97%, or 98%, or 99% identical to the amino acid sequence selected from SEQ ID NOs: 58-62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57. In embodiments, the variant ECD comprises an amino acid selected from SEQ ID NOs: 59-62.

In embodiments, the variant ECD is a recombinant fusion protein.

In aspects, the present disclosure relates to various polypeptides, and fusion proteins and chimeric proteins and chimeric proteins comprising one or more variant ECDs of the present disclosure.

### Polypeptides Comprising Variant Extracellular Domains (ECDs) of PD-1

In one aspect, the present disclosure relates to a polypeptide comprising a variant extracellular domain (ECD) of PD-1, wherein the variant ECD comprises one or more mutations (e.g., one or more insertions, deletions, substitutions, or a combination thereof) at one or more amino acid residues corresponding to one or more of the arginine residue at the position 86 (R86), the serine residue at the position 87 (S87), and glutamine residue at the position 88 (Q88) with respect to SEQ ID NO: 57, wherein the polypeptide has less affinity to pembrolizumab and/or nivolumab compared to a wild type PD-1 ECD having an amino acid sequence that is identical to the amino acid sequence of SEQ ID NO: 58.

In one aspect, the present disclosure relates to a polypeptide comprising a variant extracellular domain (ECD) of PD-1, wherein the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57, wherein the polypeptide has less affinity to pembrolizumab and/or nivolumab compared to a wild type PD-1 ECD having an amino acid sequence that is identical to the amino acid sequence of SEQ ID NO: 58.

In embodiments, the variant ECD comprises an amino acid substitution at an amino acid corresponding to R86 and S87 with respect to SEQ ID NO: 57. In embodiments, the variant ECD comprises an amino acid substitution at an amino acid corresponding to S87 and Q88 with respect to SEQ ID NO: 57. In embodiments, the variant ECD comprises an amino acid substitution at an amino acid corresponding to R86 and Q88 with respect to SEQ ID NO: 57. In embodiments, the variant ECD comprises an amino acid substitution at an amino acid corresponding to R86, S87, and Q88 with respect to SEQ ID NO: 57.

In embodiments, the variant ECD comprises an amino acid substitution at an amino acid corresponding to R86 and S87 with respect to SEQ ID NO: 57. In embodiments, the variant ECD comprises an amino acid substitution at an amino acid corresponding to S87 and Q88 with respect to SEQ ID NO: 57. In embodiments, the variant ECD comprises an amino acid substitution at an amino acid corresponding to R86 and Q88 with respect to SEQ ID NO: 57. In embodiments, the variant ECD comprises an amino acid substitution at an amino acid corresponding to R86, S87, and Q88 with respect to SEQ ID NO: 57.

In embodiments, the variant ECD and/or the polypeptide has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the variant ECD and/or the polypeptide has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2.

In embodiments, the polypeptide comprises the variant ECD comprising one or more substitutions at one or more amino acid residues corresponding to one or more of arginine residue at the position 86 (R86), serine residue at the position 87 (S87), and glutamine residue at the position 88 (Q88) with respect to SEQ ID NO: 57.

In embodiments, S87 with respect to SEQ ID NO: 57 is replaced with an amino acid residue that is aromatic, aliphatic, hydrophobic, polar, hydrophilic, neutral of charge, negatively charged, positively charged or combination thereof. In embodiments, S87 with respect to SEQ ID NO: 57 is replaced with an amino acid residue that is an hydrophilic, polar and positively charged residue is selected from lysine (K) and arginine (R). In embodiments, S87 with respect to SEQ ID NO: 57 is replaced with an amino acid residue that is an aromatic, polar and positively charged hydrophilic residue, wherein the aromatic, polar and positively charged hydrophilic residue is histidine (H). In embodiments, S87 with respect to SEQ ID NO: 57 is replaced with a hydrophilic is a polar and negatively charged hydrophilic amino acid residue selected from aspartate (D) and glutamate (E). In embodiments, S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, S87 with respect to SEQ ID NO: 57 is replaced with an hydrophilic, polar and neutral of charge amino acid residue selected from asparagine (N), glutamine (Q), threonine (T), proline (P), and cysteine (C). In embodiments, S87 with respect to SEQ ID NO: 57 is replaced with cysteine (C). In embodiments, the polypeptide comprises the variant ECD comprising a S87C substitution.

In embodiments, the R86 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the R86 with respect to SEQ ID NO: 57 is replaced with an hydrophilic, polar and positively charged residue, which is lysine. In embodiments, the R86 with respect to SEQ ID NO: 57 is replaced with an aromatic, polar and positively charged hydrophilic residue, which is histidine (H). In embodiments, the R86 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the R86 with respect to SEQ ID NO: 57 is replaced with an hydrophilic is a polar and negatively charged hydrophilic amino acid residue selected from aspartate (D) and glutamate (E). In embodiments, the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the hydrophobic, aliphatic amino acid residue is alanine (A). In embodiments, the polypeptide comprises the variant ECD comprising a R86A substitution. In embodiments, the polypeptide comprises the variant ECD comprising a R86A, S87C double substitution.

In embodiments, the Q88 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the Q88 with respect to SEQ ID NO: 57 is replaced with an hydrophilic, polar and positively charged residue selected from arginine and lysine. In embodiments, the Q88 with respect to SEQ ID NO: 57 is replaced with an aromatic, polar and positively charged hydrophilic residue, which is histidine (H). In embodiments, the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the Q88 with respect to SEQ ID NO: 57 is replaced with an hydrophilic is a polar and negatively charged hydrophilic amino acid residue selected from aspartate (D) and glutamate (E). In embodiments, the polar and negatively charged hydrophilic amino acid residue is glutamate (E). In embodiments, the polypeptide comprises the variant ECD comprising a Q88E substitution. In embodiments, the polypeptide comprises the variant ECD comprising a S87C, Q88E double substitution. In embodiments, the polypeptide comprises the variant ECD comprising a S87C, Q88E R86A triple substitution.

In embodiments, the polypeptide comprises the variant ECD comprising an amino acid sequence that is at least 95%, or 96%, or 97%, or 98%, or 99% identical to the amino acid sequence selected from SEQ ID NOs: 58-62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57. In embodiments, the polypeptide comprises the variant ECD comprising an amino acid selected from SEQ ID NOs: 59-62.

In one aspect, the present disclosure relates to a recombinant protein comprising variant extracellular domain (ECD) of PD-1 comprising an amino acid sequence that is 95% identical to the amino acids of (i) SEQ ID NO: 59, or (ii) SEQ ID NO: 60, (iii) SEQ ID NO: 61, or (iv) SEQ ID NO: 62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57.

In embodiments, the variant ECD and/or the recombinant protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the variant ECD and/or the recombinant protein has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2.

In embodiments, polypeptide is a recombinant fusion protein.

In one aspect, the present disclosure relates to a nucleic acid encoding the chimeric protein of any of the embodiments disclosed herein. In embodiments, the nucleic acid is an mRNA. In embodiments, the nucleic acid is a DNA. In one aspect, the present disclosure relates to an expression vector, comprising a nucleic acid encoding the chimeric protein of any of the embodiments disclosed herein. In one aspect, the present disclosure relates to a host cell, comprising the expression vector of any of the embodiments disclosed herein, mRNA of any of the embodiments disclosed herein, or DNA of any of the embodiments disclosed herein.

In one aspect, the present disclosure relates to a pharmaceutical composition, comprising a therapeutically effective amount of the chimeric protein of any of the embodiments disclosed herein, or the nucleic acid of any of the embodiments disclosed herein, or the expression vector of any of the embodiments disclosed herein, or the host cell of any of the embodiments disclosed herein.

### Chimeric Proteins Comprising Variant Extracellular Domains (ECDs) of PD-1

In one aspect, the present disclosure relates to a chimeric protein comprising: (a) a variant extracellular domain (ECD) of PD-1, wherein the variant ECD comprises one or more mutations (e.g., one or more insertions, deletions, substitutions, or a combination thereof) at one or more amino acid residues corresponding to one or more of the arginine residue at the position 86 (R86), the serine residue at the position 87 (S87), and glutamine residue at the position 88 (Q88) with respect to SEQ ID NO: 57; and (b) a carrier protein, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

In one aspect, the present disclosure relates to a chimeric protein comprising: (a) a variant extracellular domain (ECD) of PD-1, wherein the variant ECD comprises one or more substitutions at one or more amino acid residues corresponding to one or more of the arginine residue atthe position 86 (R86), the serine residue at the position 87 (S87), and glutamine residue at the position 88 (Q88) with respect to SEQ ID NO: 57; and (b) a carrier protein, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

It is noted that the wild type ECD of PD-1 has an amino acid sequence of SEQ ID NO: 58. Full length PD-1 sequence, which includes signal sequence, transmembrane domain, and intracellular domain, in addition to the ECD, has an amino acid sequence of SEQ ID NO: 57. While the amino acid residues of the variant ECDs that are present in the chimeric proteins disclosed herein are numbered with respect to SEQ ID NO: 57, the variant ECDs disclosed herein include only ECDs, i.e. the variant ECDs do not include signal sequence, transmembrane domain, and intracellular domain.

In embodiments, the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the affinity to pembrolizumab and/or nivolumab is assessed based on the measurement of K_{D} value as shown in Example 3. In embodiments, the affinity to pembrolizumab and/or nivolumab may be assessed using as assay including, but not limited to, ELISA, Surface plasmon resistance (SPR), and bio-layer interferometry.

In embodiments, the variant ECD has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2. In embodiments, the PD-1 ligand is selected from PD-L1 and PD-L2. In embodiments, the affinity to PD-L1 and/or PD-L2 is assessed based on the measurement of K_{D} value as shown in Example 3. In embodiments, the affinity to PD-L1 and/or PD-L2 may be assessed using as assay including, but not limited to, ELISA, Surface plasmon resistance (SPR), and bio-layer interferometry.

In embodiments, the chimeric protein comprises the variant ECD comprising one or more substitutions at one or more amino acid residues corresponding to one or more of arginine residue at the position 86 (R86), serine residue at the position 87 (S87), and glutamine residue at the position 88 (Q88) with respect to SEQ ID NO: 57.

It is noted that the wild type ECD of PD-1 has an amino acid sequence of SEQ ID NO: 58. Full length PD-1 sequence, which includes signal sequence, transmembrane domain, and intracellular domain, in addition to the ECD, has an amino acid sequence of SEQ ID NO: 57. While the amino acid residues of the variant ECDs are numbered with respect to SEQ ID NO: 57, the variant ECDs disclosed herein include only ECDs, i.e. the variant ECDs do not include signal sequence, transmembrane domain, and intracellular domain.

In embodiments, S87 with respect to SEQ ID NO: 57 is replaced with an amino acid residue that is aromatic, aliphatic, hydrophobic, polar, hydrophilic, neutral of charge, negatively charged, positively charged or combination thereof. In embodiments, S87 with respect to SEQ ID NO: 57 is replaced with an amino acid residue that is an hydrophilic, polar and positively charged residue is selected from lysine (K) and arginine (R). In embodiments, S87 with respect to SEQ ID NO: 57 is replaced with an amino acid residue that is an aromatic, polar and positively charged hydrophilic residue, wherein the aromatic, polar and positively charged hydrophilic residue is histidine (H). In embodiments, S87 with respect to SEQ ID NO: 57 is replaced with a hydrophilic is a polar and negatively charged hydrophilic amino acid residue selected from aspartate (D) and glutamate (E). In embodiments, S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, S87 with respect to SEQ ID NO: 57 is replaced with an hydrophilic, polar and neutral of charge amino acid residue selected from asparagine (N), glutamine (Q), threonine (T), proline (P), and cysteine (C). In embodiments, S87 with respect to SEQ ID NO: 57 is replaced with cysteine (C). In embodiments, the chimeric protein comprises the variant ECD comprising a S87C substitution.

In embodiments, the R86 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the R86 with respect to SEQ ID NO: 57 is replaced with an hydrophilic, polar and positively charged residue, which is lysine. In embodiments, the R86 with respect to SEQ ID NO: 57 is replaced with an aromatic, polar and positively charged hydrophilic residue, which is histidine (H). In embodiments, the R86 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the R86 with respect to SEQ ID NO: 57 is replaced with an hydrophilic is a polar and negatively charged hydrophilic amino acid residue selected from aspartate (D) and glutamate (E). In embodiments, the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the hydrophobic, aliphatic amino acid residue is alanine (A). In embodiments, the chimeric protein comprises the variant ECD comprising a R86A substitution. In embodiments, the chimeric protein comprises the variant ECD comprising a R86A, S87C double substitution.

In embodiments, the Q88 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the Q88 with respect to SEQ ID NO: 57 is replaced with an hydrophilic, polar and positively charged residue selected from arginine and lysine. In embodiments, the Q88 with respect to SEQ ID NO: 57 is replaced with an aromatic, polar and positively charged hydrophilic residue, which is histidine (H). In embodiments, the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the Q88 with respect to SEQ ID NO: 57 is replaced with an hydrophilic is a polar and negatively charged hydrophilic amino acid residue selected from aspartate (D) and glutamate (E). In embodiments, the polar and negatively charged hydrophilic amino acid residue is glutamate (E). In embodiments, the chimeric protein comprises the variant ECD comprising a Q88E substitution. In embodiments, the chimeric protein comprises the variant ECD comprising a S87C, Q88E double substitution. In embodiments, the chimeric protein comprises the variant ECD comprising a R86A, S87C, Q88E triple substitution.

In embodiments, the chimeric protein comprises the variant ECD comprising an amino acid sequence that is at least 95%, or 96%, or 97%, or 98%, or 99% identical to the amino acid sequence selected from SEQ ID NOs: 58-62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57. In embodiments, the chimeric protein comprises the variant ECD comprising an amino acid selected from SEQ ID NOs: 59-62.

In one aspect, the present disclosure relates to a chimeric protein comprising: (a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the variant extracellular domain of PD-1 comprises: an alanine residue at the position 86 corresponding to SEQ ID NO: 57; a cysteine residue at the position 87 corresponding to SEQ ID NO: 57; and/or glutamic acid at the position 88 corresponding to SEQ ID NO: 57; and (b) a carrier protein selected from selected from albumin, transferrin, an Fc, or elastin-like protein, or a variant thereof.

In embodiments, the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the variant ECD and/or the chimeric protein has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2.

In embodiments, the carrier protein is selected from albumin, transferrin, an Fc, or elastin-like protein, or a variant thereof. See, e.g., U.S. Pat. No. 9,458,218, which is hereby incorporated by reference in its entirety. In embodiments, the carrier protein an Fc or a variant thereof. See, e.g., US 2014/0113370 which is hereby incorporated by reference in its entirety In embodiments, the Fc domain is selected from an IgG Fc domain, an IgA Fc domain, an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain.

In embodiments, the Fc domain comprises hinge-CH2-CH3 Fc domain derived from IgG4. In embodiments, the Fc domain the hinge-CH2-CH3 Fc domain is derived from human IgG4. In embodiments, the Fc domain comprises an amino acid sequence that is at least 95% identical to the amino acid sequence of SEQ ID NO: 1, SEQ ID NO: 2, or SEQ ID NO: 3. In embodiments, the Fc domain comprises hinge-CH2-CH3 Fc domain derived from IgG1. In embodiments, the Fc domain the hinge-CH2-CH3 Fc domain is derived from human IgG1.

In embodiments, the chimeric protein further comprises one or more joining linkers, such joining linkers independently selected from SEQ ID NOs: 4-50. In embodiments, the albumin is human serum albumin.

In embodiments, the chimeric protein is a recombinant chimeric protein.

In one aspect, the present disclosure relates to a nucleic acid encoding the chimeric protein of any of the embodiments disclosed herein. In embodiments, the nucleic acid is an mRNA. In embodiments, the nucleic acid is a DNA.

In one aspect, the present disclosure relates to an expression vector, comprising a nucleic acid encoding the chimeric protein of any of the embodiments disclosed herein.

In one aspect, the present disclosure relates to a host cell, comprising the expression vector any of the embodiments disclosed herein, mRNA any of the embodiments disclosed herein, or DNA any of the embodiments disclosed herein.

In one aspect, the present disclosure relates to a pharmaceutical composition, comprising a therapeutically effective amount of the chimeric protein of any of the embodiments disclosed herein, or the nucleic acid any of the embodiments disclosed herein, or the expression vector any of the embodiments disclosed herein, or the host cell any of the embodiments disclosed herein.

### Chimeric Proteins Comprising Variant Extracellular Domains (ECDs) of PD-1 and a Portion of a Type II Membrane Protein

In one aspect, the present disclosure relates to a chimeric protein comprising (a) a first domain comprising a variant extracellular domain (ECD) of PD-1, wherein the variant ECD comprises one or more mutations (e.g., one or more insertions, deletions, substitutions, or a combination thereof) at one or more amino acid residues corresponding to one or more of R86, S87, and Q88 with respect to SEQ ID NO: 57, (b) a second domain comprising an extracellular domain of a transmembrane protein, and (c) a linker, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

In one aspect, the present disclosure relates to a chimeric protein comprising (a) a first domain comprising a variant extracellular domain (ECD) of PD-1, wherein the variant ECD comprises one or more substitutions at one or more amino acid residues corresponding to one or more of R86, S87, and Q88 with respect to SEQ ID NO: 57, (b) a second domain comprising an extracellular domain of a transmembrane protein, and (c) a linker, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

It is noted that the wild type ECD of PD-1 has an amino acid sequence of SEQ ID NO: 58. Full length PD-1 sequence, which includes signal sequence, transmembrane domain, and intracellular domain, in addition to the ECD, has an amino acid sequence of SEQ ID NO: 57. While the amino acid residues of the variant ECDs that are present in the chimeric proteins disclosed herein are numbered with respect to SEQ ID NO: 57, the variant ECDs disclosed herein include only ECDs, i.e. the variant ECDs do not include signal sequence, transmembrane domain, and intracellular domain.

In embodiments, the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the affinity to pembrolizumab and/or nivolumab is assessed based on the measurement of K_{D} value as shown in Example 3. In embodiments, the affinity to pembrolizumab and/or nivolumab may be assessed using as assay including, but not limited to, ELISA, Surface plasmon resistance (SPR), and bio-layer interferometry.

In embodiments, the variant ECD has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2. In embodiments, the PD-1 ligand is selected from PD-L1 and PD-L2. In embodiments, the affinity to PD-L1 and/or PD-L2 is assessed based on the measurement of K_{D} value as shown in Example 3. In embodiments, the affinity to PD-L1 and/or PD-L2 may be assessed using as assay including, but not limited to, ELISA, Surface plasmon resistance (SPR), and bio-layer interferometry.

In embodiments, the chimeric protein comprises the variant ECD comprising one or more substitutions at one or more amino acid residues corresponding to one or more of arginine residue at the position 86 (R86), serine residue at the position 87 (S87), and glutamine residue at the position 88 (Q88) with respect to SEQ ID NO: 57. In embodiments, the variant ECD comprises an amino acid substitution at an amino acid corresponding to R86 and S87 with respect to SEQ ID NO: 57. In embodiments, the variant ECD comprises an amino acid substitution at an amino acid corresponding to S87 and Q88 with respect to SEQ ID NO: 57. In embodiments, the variant ECD comprises an amino acid substitution at an amino acid corresponding to R86 and Q88 with respect to SEQ ID NO: 57. In embodiments, the variant ECD comprises an amino acid substitution at an amino acid corresponding to R86, S87, and Q88 with respect to SEQ ID NO: 57.

In embodiments, S87 with respect to SEQ ID NO: 57 is replaced with an amino acid residue that is aromatic, aliphatic, hydrophobic, polar, hydrophilic, neutral of charge, negatively charged, positively charged or combination thereof. In embodiments, S87 with respect to SEQ ID NO: 57 is replaced with an amino acid residue that is an hydrophilic, polar and positively charged residue is selected from lysine (K) and arginine (R). In embodiments, S87 with respect to SEQ ID NO: 57 is replaced with an amino acid residue that is an aromatic, polar and positively charged hydrophilic residue, wherein the aromatic, polar and positively charged hydrophilic residue is histidine (H). In embodiments, S87 with respect to SEQ ID NO: 57 is replaced with a hydrophilic is a polar and negatively charged hydrophilic amino acid residue selected from aspartate (D) and glutamate (E). In embodiments, S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, S87 with respect to SEQ ID NO: 57 is replaced with an hydrophilic, polar and neutral of charge amino acid residue selected from asparagine (N), glutamine (Q), threonine (T), proline (P), and cysteine (C). In embodiments, S87 with respect to SEQ ID NO: 57 is replaced with cysteine (C). In embodiments, the chimeric protein comprises the variant ECD comprising a S87C substitution.

In embodiments, the R86 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the R86 with respect to SEQ ID NO: 57 is replaced with an hydrophilic, polar and positively charged residue, which is lysine. In embodiments, the R86 with respect to SEQ ID NO: 57 is replaced with an aromatic, polar and positively charged hydrophilic residue, which is histidine (H). In embodiments, the R86 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the R86 with respect to SEQ ID NO: 57 is replaced with an hydrophilic is a polar and negatively charged hydrophilic amino acid residue selected from aspartate (D) and glutamate (E). In embodiments, the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the hydrophobic, aliphatic amino acid residue is alanine (A). In embodiments, the chimeric protein comprises the variant ECD comprising a R86A substitution. In embodiments, the chimeric protein comprises the variant ECD comprising a R86A, S87C double substitution.

In embodiments, the Q88 with respect to SEQ ID NO: 57 is replaced with an amino acid residue that is aromatic, aliphatic, hydrophobic, polar, hydrophilic, neutral of charge, negatively charged, positively charged or combination thereof. In embodiments, the Q88 with respect to SEQ ID NO: 57 is replaced with an hydrophilic, polar and positively charged residue selected from arginine and lysine. In embodiments, the Q88 with respect to SEQ ID NO: 57 is replaced with an aromatic, polar and positively charged hydrophilic residue, which is histidine (H). In embodiments, the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C). In embodiments, the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y). In embodiments, the Q88 with respect to SEQ ID NO: 57 is replaced with an hydrophilic is a polar and negatively charged hydrophilic amino acid residue selected from aspartate (D) and glutamate (E). In embodiments, the polar and negatively charged hydrophilic amino acid residue is glutamate (E). In embodiments, the chimeric protein comprises the variant ECD comprising a Q88E substitution. In embodiments, the chimeric protein comprises the variant ECD comprising a S87C, Q88E double substitution. In embodiments, the chimeric protein comprises the variant ECD comprising a R86A, S87C, Q88E triple substitution.

In embodiments, the chimeric protein comprises the variant ECD comprising an amino acid sequence that is at least 95%, or 96%, or 97%, or 98%, or 99% identical to the amino acid sequence selected from SEQ ID NOs: 58-62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57. In embodiments, the chimeric protein comprises the variant ECD comprising an amino acid selected from SEQ ID NOs: 59-62.

In one aspect, the present disclosure relates to a chimeric protein comprising (a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the variant extracellular domain of PD-1 comprises: an alanine residue at the position 86 corresponding to SEQ ID NO: 57; a cysteine residue at the position 87 corresponding to SEQ ID NO: 57; and/or a glutamic acid at the position 88 corresponding to SEQ ID NO: 57; (b) a second domain comprising an extracellular domain of a Type II transmembrane protein selected from 4-1BBL, OX40L, CD70, CD30L, CD40L, GITRL, TL1A, and LIGHT, and (c) a linker. In embodiments, the heterologous chimeric protein comprises a general structure of: N terminus-(a)-(c)-(b)-C terminus, wherein: (c) is the linker, and (b) is the second domain comprising an extracellular domain of Type II transmembrane protein.

In embodiments, the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58. In embodiments, the variant ECD and/orthe chimeric protein has an affinity to a PD-1 ligand that is equivalent to the affinity of the wild type PD-1 ECD having an amino acid sequence that is identical to the amino acid sequence of SEQ ID NO: 58. In embodiments, the PD-1 ligand is selected from PD-L1 and PD-L2.

In embodiments, the transmembrane protein is a portion of a Type II transmembrane protein. In embodiments, the Type II transmembrane protein is selected from 4-1BBL, OX40L, CD70, CD30L, CD40L, GITRL, TL1A, and LIGHT.

In embodiments, the transmembrane protein is a portion of 4-1BBL. In embodiments, the portion of 4-1BBL is a portion of the extracellular domain of 4-1BBL. In embodiments, the present chimeric protein further comprises a domain, e.g., the extracellular domain, of the immune stimulatory molecule 4-1BB ligand (4-1BBL). 4-1BBL is a type II transmembrane protein belonging to the Tumor Necrosis Factor (TNF) superfamily.

In embodiments, the second domain is a portion of 4-1BBL. In embodiments, the second domain comprises substantially all the extracellular domain of 4-1BBL. In embodiments, the second domain is capable of binding 4-1BB (also known as cluster of differentiation 137 (CD137) or tumor necrosis factor ligand superfamily member 9 (TNFSF9)). In embodiments, the binding to 4-1BB increases or activates an immune stimulatory signal. In embodiments, the binding to 4-1BB costimulates CD4 and/or CD8 T-cells. 4-1BBL is also known as cluster of differentiation 137 ligand (CD137L). Thus, throughout this disclosure, 4-1BBL and CD137L are synonymous, when referenced alone and/or when referenced in context of a chimeric protein. 4-1BB ligand (4-1BBL) binds to the 4-1BB receptor on activated T Lymphocytes and antigen-presenting cells (APC). 4-1BB signaling is believed to follow an immune synapse, formed by 4-1BB+ lymphocytes and 4-1BBL+ antigen-presenting cells. For example, 4-1BBL binding induces B cell proliferation and immunoglobulin production. T cells are the major 4-1BB-expressing cells and may engage 4-1BBL on macrophages and or APCs for their activation. CD8+ T cells release IL-13 as well as IFN-γ through 4-1BB signaling.

In embodiments, the present chimeric protein comprises a domain, e.g., the extracellular domain, of human 4-1BBL. The human 4-1BBL comprises the following amino acid sequence:

(SEQ ID NO: 72)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

The amino acid sequence of extracellular domain human 4-1BBL (amino acids 50-254 of SEQ ID NO: 72) is the following:

(SEQ ID NO: 64)
ACPWAVSGARASPGSAASPRLREGPELSPDDPAGLLDLRQGMFAQLVAQN
VLLIDGPLSWYSDPGLAGVSLTGGLSYKEDTKELVVAKAGVYYVFFQLEL
RRVVAGEGSGSVSLALHLQPLRSAAGAAALALTVDLPPASSEARNSAFGF
QGRLLHLSAGQRLGVHLHTEARARHAWQLTQGATVLGLFRVTPEIPAGLP
SPRSE

In embodiments, the present chimeric protein comprises the extracellular domain of human 4-1BBL which has the amino acid sequence of SEQ ID NO: 64. In embodiments, the present variant ECDs of PD-1 and chimeric proteins comprising the variant ECDs may comprise the extracellular domain of 4-1BBL as described herein, or a variant or functional fragment thereof. For instance, the chimeric protein may comprise a sequence of the extracellular domain of 4-1BBL as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of 4-1BBL as described herein.

4-1BBL derivatives can be constructed from available structural data, including that described by Won et al., “The structure of the trimer of human 4-1BB ligand is unique among members of the tumor necrosis factor superfamily.” J. Biol. Chem. 285: 9202-9210 (2010); Gilbreth et al., “Crystal structure of the human 4-1BB/4-1BBL complex.” J Biol Chem 293: 9880-9891 (2018); and Bitra et al., “Crystal structures of the human 4-1BB receptor bound to its ligand 4-1BBL reveal covalent receptor dimerization as a potential signaling amplifier.” J Biol Chem 293: 9958-9969 (2018).

In embodiments, the present variant ECDs of PD-1 and chimeric proteins comprising the variant ECDs may comprise a variant extracellular domain of 4-1BBL in which the signal peptide (e.g., as provided in SEQ ID NO: 64) is replaced with an alternative signal peptide. In embodiments, the present chimeric protein may comprise a variant extracellular domain of 4-1BBL which is expressed from a cDNA that has been codon-optimized for expression in protein producing cells such as Chinese Hamster Ovary (CHO) or HEK cells.

In embodiments, the extracellular domain of 4-1BBL refers to a portion of protein which is capable of interacting with the extracellular environment. In embodiments, the extracellular domain of 4-1BBL is the entire amino acid sequence of the protein which is external of a cell or the cell membrane. In embodiments, the extracellular domain of 4-1BBL is a portion of an amino acid sequence of the protein which is external of a cell or the cell membrane and is needed for signal transduction and/or ligand binding as may be assayed using methods know in the art.

In embodiments, the extracellular domain of 4-1BBL refers to a portion of the protein which is capable for binding to the 4-1BB receptor. Similar to other TNF superfamily members, membrane-bound 4-1BBL exists as a homotrimer. 4-1BBL binds to 4-1BB, a member of the TNF receptor superfamily that is expressed predominantly on antigen presenting cells.

In embodiments, the chimeric protein of the invention binds to human 4-1BB with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human 4-1BB with a K_{D} of less than about 1 nM, about 900 μM, about 800 μM, about 700 μM, about 600 μM, about 500 μM, about 400 μM, about 300 μM, about 200 μM, about 100 μM, about 90 μM, about 80 μM, about 70 μM, about 60 μM about 55 μM about 50 μM about 45 μM, about 40 μM, about 35 μM, about 30 μM, about 25 μM, about 20 μM, about 15 μM, or about 10 μM, or about 1 μM (as measured, for example, by surface plasmon resonance or biolayer interferometry). In embodiments, the chimeric protein binds to human 4-1BB with a K_{D} of from about 300 μM to about 700 μM.

In embodiments, the second domain is 4-1BBL, wherein the 4-1BBL is capable of binding to a 4-1BBL receptor. In embodiments, the 4-1BBL receptor is 4-1BB. In embodiments, the second domain comprises an amino acid sequence that is at least 95% identical to the amino acid sequence of SEQ ID NO: 64.

In embodiments, where a chimeric protein comprises a variant extracellular domain (ECD) of PD-1, one joining linker preceding an Fc domain, a second joining linker following the Fc domain, and an ECD of 4-1BBL, the chimeric protein may comprise the following structure:
Variant ECD of PD-1-Joining Linker 1-Fc Domain-Joining Linker 2-ECD of 4-1BBL

In embodiments, the transmembrane protein is a portion of OX40L. In embodiments, the present chimeric protein further comprises a domain, e.g., the extracellular domain, of the immune stimulatory molecule OX40 ligand (OX40L). OX40L is a type II transmembrane glycoprotein belonging to the Tumor Necrosis Factor (TNF) superfamily. Specifically, the human OX40L protein comprises 183 amino acids including an amino-terminal cytoplasmic domain (amino acids 1-23) and a carboxy-terminal extracellular domain (amino acids 51-183).

(SEQ ID NO: 73)
MERVQPLEENVGNAARPRFERNKLLLVASVIQGLGLLLCFTYICLHFSAL
QVSHRYPRIQSIKVQFTEYKKEKGFILTSQKEDEIMKVQNNSVIINCDGF
YLISLKGYFSQEVNISLHYQKDEEPLFQLKKVRSVNSLMVASLTYKDKVY
LNVTTDNTSLDDFHVNGGELILIHQNPGEFCVL

In embodiments, the present chimeric protein comprises the extracellular domain of human OX40L. The human OX40L comprises the amino acid sequence of SEQ ID NO: 73 (with the amino acid sequence of the extracellular domain comprising SEQ ID NO: 65):

(SEQ ID NO: 65)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL

In embodiments, the present variant ECDs of PD-1 and chimeric proteins comprising the variant ECDs may comprise the extracellular domain of OX40L as described herein (e.g., SEQ ID NO: 65), or a variant or functional fragment thereof. For instance, the chimeric protein may comprise a sequence of the extracellular domain of OX40L as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of OX40L as described herein.

OX40L derivatives can be constructed from available structural data, including that described by Compaan and Hymowitz, The Crystal Structure of the Costimulatory OX40-OX40L Complex Structure 14: 1321-1330. (2006).

In embodiments, the present variant ECDs of PD-1 and chimeric proteins comprising the variant ECDs may comprise a variant extracellular domain of OX40L in which the signal peptide (e.g., as provided in SEQ ID NO: 73) is replaced with an alternative signal peptide. In embodiments, the present chimeric protein may comprise a variant extracellular domain of OX40L which is expressed from a cDNA that has been codon-optimized for expression in protein producing cells such as CHO or HEK cells.

In embodiments, the extracellular domain of OX40L refers to a portion of protein which is capable of interacting with the extracellular environment. In embodiments, the extracellular domain of OX40L is the entire amino acid sequence of the protein which is external of a cell or the cell membrane. In embodiments, the extracellular domain of OX40L is a portion of an amino acid sequence of the protein which is external of a cell or the cell membrane and is needed for signal transduction and/or ligand binding as may be assayed using methods know in the art.

In embodiments, the extracellular domain of OX40L refers to a portion of the protein which is capable for binding to the OX40 receptor. Similar to other TNF superfamily members, membrane-bound OX40L exists as a homotrimer. OX40L binds to OX40, a member of the TNF receptor superfamily that is expressed predominantly on CD4+ and/or CD8+ T cells as well as a number of lymphoid and non-lymphoid cells. Evidence suggests that the major function of the OX40-OX40L interaction is to transmit a late co-stimulatory signal to promote the survival and proliferation of activated T cells and prolong immune responses.

In embodiments, the chimeric protein of the invention binds to human OX40 with a K_{D} of less than about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human OX40 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human OX40 with a K_{D} of from about 200 pM to about 600 pM (as measured, for example, by surface plasmon resonance or biolayer interferometry).

In embodiments, the chimeric protein may comprise an amino acid sequence having one or more amino acid mutations relative to any of the protein sequences described herein. In embodiments, the chimeric protein comprises a sequence that has about 1, 2, 3, 4, 5, 6, 7, 8, 9, 10, 15, 20, 25, 30, 35, 40, 45, 50, 55, 60, 65, 70, 75, 80, 85, 90, 95, or 100 or more amino acid mutations with respect to any one of the amino acid sequences of chimeric proteins disclosed herein.

In embodiments, the second domain is OX40L, wherein the OX40L is capable of binding to an OX40L receptor. In embodiments, the OX40L receptor is OX40. In embodiments, the second domain comprises an amino acid sequence that is at least 95% identical to the amino acid sequence of SEQ ID NO: 65.

In embodiments, where a chimeric protein comprises a variant extracellular domain (ECD) of PD-1, one joining linker preceding an Fc domain, a second joining linker following the Fc domain, and an ECD of OX40L, the chimeric protein may comprise the following structure:
Variant ECD of PD-1-Joining Linker 1-Fc Domain-Joining Linker 2-ECD of OX40L

In embodiments, the second domain comprises CD70. CD70 is a cytokine, which is the ligand for CD27. The CD70-CD27 pathway plays an important role in the generation and maintenance of T cell immunity, in particular during antiviral responses. Upon CD27 binding, induces the proliferation of costimulated T-cells and enhances the generation of cytolytic T-cells. In embodiments, the portion of CD70 is a portion of the extracellular domain of CD70. In embodiments, the present chimeric protein further comprises a domain, e.g., the extracellular domain, of the immune stimulatory molecule CD70. CD70 is a type II transmembrane protein.

In embodiments, the second domain is a portion of CD70. In embodiments, the second domain comprises substantially all the extracellular domain of CD70. In embodiments, the second domain is capable of binding CD27. In embodiments, the binding to CD27 increases or activates an immune stimulatory signal. In embodiments, the binding to CD27 costimulates CD4 and/or CD8 T-cells.

CD27 ligand (CD70) binds to the CD27 receptor on activated T Lymphocytes and antigen-presenting cells (APC). CD27 signaling is believed to follow an immune synapse, formed by CD27+lymphocytes and CD70+antigen-presenting cells. For example, CD70 binding induces B cell proliferation and immunoglobulin production. T cells are the major CD27-expressing cells and may engage CD70 on macrophages and or APCs for their activation. CD8+ T cells release IL-13 as well as IFN-γ through CD27 signaling.

In embodiments, the present chimeric protein comprises a domain, e.g., the extracellular domain, of human CD70. The human CD70 comprises the following amino acid sequence:

(SEQ ID NO: 74)
MPEEGSGCSVRRRPYGCVLRAALVPLVAGLVICLVVCIQRFAQAQQQLP
LESLGWDVAELQLNHTGPQQDPRLYWQGGPALGRSFLHGPELDKGQLRI
HRDGIYMVHIQVTLAICSSTTASRHHPTTLAVGICSPASRSISLLRLSF
HQGCTIASQRLTPLARGDTLCTNLTGTLLPSRNTDETFFGVQWVRP

The amino acid sequence of extracellular domain human CD70 (amino acids 50-254 of SEQ ID NO: 74) is the following:

(SEQ ID NO: 66)
QRFAQAQQQLPLESLGWDVAELQLNHTGPQQDPRLYWQGGPALGRSFLH
GPELDKGQLRIHRDGIYMVHIQVTLAICSSTTASRHHPTTLAVGICSPA
SRSISLLRLSFHQGCTIASQRLTPLARGDTLCTNLTGTLLPSRNTDETF
FGVQWVRP

In embodiments, the present chimeric protein comprises the extracellular domain of human CD70 which has the amino acid sequence of SEQ ID NO: 66. In embodiments, the present variant ECDs of PD-1 and chimeric proteins comprising the variant ECDs may comprise the extracellular domain of CD70 as described herein, or a variant or functional fragment thereof. For instance, the chimeric protein may comprise a sequence of the extracellular domain of CD70 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of CD70 as described herein.

In embodiments, the present variant ECDs of PD-1 and chimeric proteins comprising the variant ECDs may comprise a variant extracellular domain of CD70 in which the signal peptide (e.g., as provided in SEQ ID NO: 66) is replaced with an alternative signal peptide. In embodiments, the present chimeric protein may comprise a variant extracellular domain of CD70 which is expressed from a cDNA that has been codon-optimized for expression in protein producing cells such as Chinese Hamster Ovary (CHO) or HEK cells.

In embodiments, the extracellular domain of CD70 refers to a portion of protein which is capable of interacting with the extracellular environment. In embodiments, the extracellular domain of CD70 is the entire amino acid sequence of the protein which is external of a cell or the cell membrane. In embodiments, the extracellular domain of CD70 is a portion of an amino acid sequence of the protein which is external of a cell or the cell membrane and is needed for signal transduction and/or ligand binding as may be assayed using methods know in the art.

In embodiments, the extracellular domain of CD70 refers to a portion of the protein which is capable for binding to the CD27 receptor. In embodiments, the chimeric protein of the invention binds to human CD27 with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CD27 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human CD27 with a K_{D} of from about 300 pM to about 700 pM.

In embodiments, the second domain is CD70, wherein the CD70 is capable of binding to a CD70 receptor. In embodiments, the CD70 receptor is CD27. In embodiments, the second domain comprises an amino acid sequence that is at least 95% identical to the amino acid sequence of SEQ ID NO: 66.

In embodiments, where a chimeric protein comprises a variant extracellular domain (ECD) of PD-1, one joining linker preceding an Fc domain, a second joining linker following the Fc domain, and an ECD of CD70, the chimeric protein may comprise the following structure:
Variant ECD of PD-1-Joining Linker 1-Fc Domain-Joining Linker 2-ECD of CD70

In embodiments, the present chimeric protein further comprises a domain, e.g., the extracellular domain, of the immune stimulatory molecule CD30 ligand (CD30L, also known as CD154). CD30L is a type II transmembrane protein belonging to the Tumor Necrosis Factor (TNF) superfamily. CD30L expressed on activated T cells, B cells, monocytes and granulocytes. CD30 has been described as a marker of memory T cells but can also be expressed by activated B cells and effector T cells. CD30 ligation by CD30L mediates pleiotropic effects including cell proliferation, activation, differentiation and cell death by apoptosis

In embodiments, the second domain is a portion of CD30L. In embodiments, the portion of CD30L is a portion of the extracellular domain of CD30L. In embodiments, the second domain comprises substantially all the extracellular domain of CD30L. In embodiments, the second domain is capable of binding CD30 (also known as Ki-1 antigen or tumor necrosis factor ligand superfamily member 8 (TNFSF8)). In embodiments, the binding to CD30 increases or activates an immune stimulatory signal. In embodiments, the binding to CD30 enhances T-cell activation, proliferation and/or cytokine production. CD30L is also known as cluster of differentiation 153 (CD153). Thus, throughout this disclosure, CD30L and CD153 are synonymous, when referenced alone and/or when referenced in context of a chimeric protein, thus, for example, variant PD-1-Fc-CD30L is the same chimeric protein as variant PD-1-Fc-CD153.

In embodiments, the present chimeric protein comprises a domain, e.g., the extracellular domain, of human CD30L. The human CD30L comprises the following amino acid sequence:

(SEQ ID NO: 75)
MDPGLQQALNGMAPPGDTAMHVPAGSVASHLGTTSRSYFYLTTATLALC
LVFTVATIMVLVVQRTDSIPNSPDNVPLKGGNCSEDLLCILKRAPFKKS
WAYLQVAKHLNKTKLSWNKDGILHGVRYQDGNLVIQFPGLYFIICQLQF
LVQCPNNSVDLKLELLINKHIKKQALVTVCESGMQTKHVYQNLSQFLLD
YLQVNTTISVNVDTFQYIDTSTFPLENVLSIFLYSNSD

The amino acid sequence of extracellular domain human CD30L (amino acids 63-234 of SEQ ID NO: 75) is the following:

(SEQ ID NO: 67)
QRTDSIPNSPDNVPLKGGNCSEDLLCILKRAPFKKSWAYLQVAKHLNKT
KLSWNKDGILHGVRYQDGNLVIQFPGLYFIICQLQFLVQCPNNSVDLKL
ELLINKHIKKQALVTVCESGMQTKHVYQNLSQFLLDYLQVNTTISVNVD
TFQYIDTSTFPLENVLSIFLYSNSD

In embodiments, the present chimeric protein comprises the extracellular domain of human CD30L which has the amino acid sequence of SEQ ID NO: 67. In embodiments, the present variant ECDs of PD-1 and chimeric proteins comprising the variant ECDs may comprise the extracellular domain of CD30L as described herein, or a variant or functional fragment thereof. For instance, the chimeric protein may comprise a sequence of the extracellular domain of CD30L as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of CD30L as described herein.

In embodiments, the present variant ECDs of PD-1 and chimeric proteins comprising the variant ECDs may comprise a variant extracellular domain of CD30L in which the signal peptide (e.g., as provided in SEQ ID NO: 75) is replaced with an alternative signal peptide. In embodiments, the present chimeric protein may comprise a variant extracellular domain of CD30L which is expressed from a cDNA that has been codon-optimized for expression in protein producing cells such as Chinese Hamster Ovary (CHO) or HEK cells.

In embodiments, the extracellular domain of CD30L refers to a portion of protein which is capable of interacting with the extracellular environment. In embodiments, the extracellular domain of CD30L is the entire amino acid sequence of the protein which is external of a cell or the cell membrane. In embodiments, the extracellular domain of CD30L is a portion of an amino acid sequence of the protein which is external of a cell or the cell membrane and is needed for signal transduction and/or ligand binding as may be assayed using methods know in the art.

In embodiments, the extracellular domain of CD30L refers to a portion of the protein which is capable for binding to the CD30 receptor. Similar to other TNF superfamily members, membrane-bound CD30L exists as a homotrimer. CD30L binds to CD30, a member of the TNF receptor superfamily.

In embodiments, the chimeric protein of the invention binds to human CD30 with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CD30 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human CD30 with a K_{D} of from about 300 pM to about 700 pM.

In embodiments, the second domain comprises a portion of CD40L. CD40L is a cytokine, which acts as a ligand to CD40/TNFRSF5. CD40L costimulates T-cell proliferation and cytokine production. Its cross-linking on T-cells generates a costimulatory signal which enhances the production of IL-4 and IL-10 in conjunction with the TCR/CD3 ligation and CD28 costimulation. CD40L induces the activation of NF-kappa-B and the kinases MAPK8 and PAK2 in T-cells. In embodiments, the portion of CD40L is a portion of the extracellular domain of CD40L. In embodiments, the present chimeric protein further comprises a domain, e.g., the extracellular domain, of the immune stimulatory molecule CD40L. CD40L is a type II transmembrane protein.

In embodiments, the second domain is a portion of CD40L. In embodiments, the second domain comprises substantially all the extracellular domain of CD40L. In embodiments, the second domain is capable of binding CD40. In embodiments, the binding to CD40 increases or activates an immune stimulatory signal. In embodiments, the binding to CD40 costimulates CD4 and/or CD8 T-cells.

In embodiments, the present chimeric protein comprises a domain, e.g., the extracellular domain, of human CD40L. The human CD40L comprises the following amino acid sequence:

(SEQ ID NO: 76)
MIETYNQTSPRSAATGLPISMKIFMYLLTVFLITQMIGSALFAVYLHRR
LDKIEDERNLHEDFVFMKTIQRCNTGERSLSLLNCEEIKSQFEGFVKDI
MLNKEETKKENSFEMQKGDQNPQIAAHVISEASSKTTSVLQWAEKGYYT
MSNNLVTLENGKQLTVKRQGLYYIYAQVTFCSNREASSQAPFIASLCLK
SPGRFERILLRAANTHSSAKPCGQQSIHLGGVFELQPGASVFVNVTDPS
QVSHGTGFTSFGLLKL

The amino acid sequence of extracellular domain human CD40L (amino acids 50-254 of SEQ ID NO: 76) is the following:

(SEQ ID NO: 68)
HRRLDKIEDERNLHEDFVFMKTIQRCNTGERSLSLLNCEEIKSQFEGFV
KDIMLNKEETKKENSFEMQKGDQNPQIAAHVISEASSKTTSVLQWAEKG
YYTMSNNLVTLENGKQLTVKRQGLYYIYAQVTFCSNREASSQAPFIASL
CLKSPGRFERILLRAANTHSSAKPCGQQSIHLGGVFELQPGASVFVNVT
DPSQVSHGTGFTSFGLLKL

In embodiments, the present chimeric protein comprises the extracellular domain of human CD40L which has the amino acid sequence of SEQ ID NO: 68. In embodiments, the present variant ECDs of PD-1 and chimeric proteins comprising the variant ECDs may comprise the extracellular domain of CD40L as described herein, or a variant or functional fragment thereof. For instance, the chimeric protein may comprise a sequence of the extracellular domain of CD40L as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of CD40L as described herein.

OX40L derivatives can be constructed from available structural data, including that described by Karpusas et al., 2 A crystal structure of an extracellular fragment of human CD40 ligand, Structure 3:1031-1039 (1995); Karpusas et al., Structure of CD40 ligand in complex with the Fab fragment of a neutralizing humanized antibody, Structure 9: 321-329 (2001); Silvian et al., Small Molecule Inhibition of the TNF Family Cytokine CD40 Ligand through a Subunit Fracture Mechanism, ACS Chem Biol 6: 636-647 (2011); An et al., Crystallographic and mutational analysis of the CD40-CD154 complex and its implications for receptor activation J Biol Chem 286: 11226-11235 (2011).

In embodiments, the present variant ECDs of PD-1 and chimeric proteins comprising the variant ECDs may comprise a variant extracellular domain of CD40L in which the signal peptide (e.g., as provided in SEQ ID NO: 68) is replaced with an alternative signal peptide. In embodiments, the present chimeric protein may comprise a variant extracellular domain of CD40L which is expressed from a cDNA that has been codon-optimized for expression in protein producing cells such as Chinese Hamster Ovary (CHO) or HEK cells.

In embodiments, the extracellular domain of CD40L refers to a portion of protein which is capable of interacting with the extracellular environment. In embodiments, the extracellular domain of CD40L is the entire amino acid sequence of the protein which is external of a cell or the cell membrane. In embodiments, the extracellular domain of CD40L is a portion of an amino acid sequence of the protein which is external of a cell or the cell membrane and is needed for signal transduction and/or ligand binding as may be assayed using methods know in the art.

In embodiments, the extracellular domain of CD40L refers to a portion of the protein which is capable for binding to the CD40 receptor. In embodiments, the chimeric protein of the invention binds to human CD40 with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CD40 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human CD40 with a K_{D} of from about 300 μM to about 700 μM.

In embodiments, the second domain is CD40L, wherein the CD40L is capable of binding to a CD40L receptor. In embodiments, the CD40L receptor is CD40. In embodiments, the second domain comprises an amino acid sequence that is at least 95% identical to the amino acid sequence of SEQ ID NO: 68.

In embodiments, where a chimeric protein comprises a variant extracellular domain (ECD) of PD-1, one joining linker preceding an Fc domain, a second joining linker following the Fc domain, and an ECD of CD40L, the chimeric protein may comprise the following structure:
Variant ECD of PD-1-Joining Linker 1-Fc Domain-Joining Linker 2-ECD of CD40L

In embodiments, the second domain comprises a portion of GITRL. GITRL member of tumor necrosis factor ligand superfamily (tumor necrosis factor ligand superfamily member 18). GITRL is a cytokine that binds to TNFRSF18/AITR/GITR, and regulates T-cell responses. GITRL can function as a costimulator and lower the threshold for T-cell activation and T-cell proliferation. GITRL is important for interactions between activated T-lymphocytes and endothelial cells. In embodiments, the portion of GITRL is a portion of the extracellular domain of GITRL. In embodiments, the present chimeric protein further comprises a domain, e.g., the extracellular domain, of the immune stimulatory molecule GITRL. GITRL is a type II transmembrane protein.

In embodiments, the second domain is a portion of GITRL. In embodiments, the second domain comprises substantially all the extracellular domain of GITRL. In embodiments, the second domain is capable of binding GITR. In embodiments, the present chimeric protein comprises a domain, e.g., the extracellular domain, of human GITRL. The human GITRL comprises the following amino acid sequence:

(SEQ ID NO: 77)
MIETYNQTSPRSAATGLPISMKIFMYLLTVFLITQMIGSALFAVYLHRR
LDKIEDERNLHEDFVFMKTIQRCNTMTLHPSPITCEFLFSTALISPKMC
LSHLENMPLSHSRTQGAQRSSWKLWLFCSIVMLLFLCSFSWLIFIFLQL
ETAKEPCMAKFGPLPSKWQMASSEPPCVNKVSDWKLEILQNGLYLIYGQ
VAPNANYNDVAPFEVRLYKNKDMIQTLTNKSKIQNVGGTYELHVGDTID
LIFNSEHQVLKNNTYWGIILLANPQFIS

The amino acid sequence of extracellular domain human GITRL (amino acids 50-254 of SEQ ID NO: 77) is the following:

(SEQ ID NO: 69)
QLETAKEPCMAKFGPLPSKWQMASSEPPCVNKVSDWKLEILQNGLYLIY
GQVAPNANYNDVAPFEVRLYKNKDMIQTLTNKSKIQNVGGTYELHVGDT
IDLIFNSEHQVLKNNTYWGIILLANPQFIS

In embodiments, the present chimeric protein comprises the extracellular domain of human GITRL which has the amino acid sequence of SEQ ID NO: 69. In embodiments, the present variant ECDs of PD-1 and chimeric proteins comprising the variant ECDs may comprise the extracellular domain of GITRL as described herein, or a variant or functional fragment thereof. For instance, the chimeric protein may comprise a sequence of the extracellular domain of GITRL as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of GITRL as described herein.

OX40L derivatives can be constructed from available structural data, including that described by Chattopadhyay et al., Assembly and structural properties of glucocorticoid-induced TNF receptor ligand: Implications for function, Proc Natl Acad Sci USA 104: 19452-19457 (2007).

In embodiments, the present variant ECDs of PD-1 and chimeric proteins comprising the variant ECDs may comprise a variant extracellular domain of GITRL in which the signal peptide (e.g., as provided in SEQ ID NO: 69) is replaced with an alternative signal peptide. In embodiments, the present chimeric protein may comprise a variant extracellular domain of GITRL which is expressed from a cDNA that has been codon-optimized for expression in protein producing cells such as Chinese Hamster Ovary (CHO) or HEK cells.

In embodiments, the extracellular domain of GITRL refers to a portion of protein which is capable of interacting with the extracellular environment. In embodiments, the extracellular domain of GITRL is the entire amino acid sequence of the protein which is external of a cell or the cell membrane. In embodiments, the extracellular domain of GITRL is a portion of an amino acid sequence of the protein which is external of a cell or the cell membrane and is needed for signal transduction and/or ligand binding as may be assayed using methods know in the art.

In embodiments, the extracellular domain of GITRL refers to a portion of the protein which is capable for binding to the GITR receptor. In embodiments, the chimeric protein of the invention binds to human GITR with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human GITR with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human GITR with a K_{D} of from about 300 μM to about 700 μM.

In embodiments, the second domain is GITRL, wherein the GITRL is capable of binding to a GITRL receptor. In embodiments, the GITRL receptor is GITR. In embodiments, the second domain comprises an amino acid sequence that is at least 95% identical to the amino acid sequence of SEQ ID NO: 69.

In embodiments, where a chimeric protein comprises a variant extracellular domain (ECD) of PD-1, one joining linker preceding an Fc domain, a second joining linker following the Fc domain, and an ECD of GITRL, the chimeric protein may comprise the following structure:
Variant ECD of PD-1-Joining Linker 1-Fc Domain-Joining Linker 2-ECD of GITRL

In embodiments, the chimeric proteins of the present invention comprise variants of the extracellular domain, which includes the receptor-binding domain, of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of TL1A, e.g., human TL1A.

In embodiments, the extracellular domain of TL1A has the following amino acid sequence:

(SEQ ID NO: 70)
SQLRAQGEACVQFQALKGQEFAPSHQQVYAPLRADGDKPRAHLTVVRQT
PTQHFKNQFPALHWEHELGLAFTKNRMNYTNKFLLIPESGDYFIYSQVT
FRGMTSECSEIRQAGRPNKPDSITVVITKVTDSYPEPTQLLMGTKSVCE
VGSNWFQPIYLGAMFSLQEGDKLMVNVSDISLVDYTKEDKTFFGAFLL

In embodiments, a chimeric protein comprises a variant of the extracellular domain of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 70.

In embodiments, the second domain of a chimeric protein comprises an amino acid sequence that is at least 95% identical to the amino acid sequence of SEQ ID NO: 70.

One of ordinary skill may select variants of the known amino acid sequence of TL1A by consulting the literature, e.g., Yue et al., “TL1, a novel tumor necrosis factor-like cytokine, induces apoptosis in endothelial cells. Involvement of activation of stress protein kinases (stress-activated protein kinase and p38 mitogen-activated protein kinase) and caspase-3-like protease.” J. Biol. Chem. 274 (3), 1479-1486 (1999); Richard et al., “Reduced monocyte and macrophage TNFSF15/TL1A expression is associated with susceptibility to inflammatory bowel disease.” PLoS Genet. 14 (9), e1007458 (2018); Migone et al., “TL1A is a TNF-like ligand for DR3 and TR6/DcR3 and functions as a T cell costimulator.” Immunity 16:479-492(2002); Jin et al., “X-ray crystal structure of TNF ligand family member TL1A at 2.1A.” Biochem. Biophys. Res. Commun. 364:1-6(2007); Zhan et al., “Decoy strategies: the structure of TL1A:DcR3 complex.” Structure 19:162-171(2011); Khan et al., “TL1A-Ig induces transplantation tolerance”, J Immunol May 1, 2013, 190 (1 Supplement) 113.2; Khan et al., “Cloning, Expression, and Functional Characterization of TL1A-lg” J Immunol Feb. 15, 2013, 190 (4) 1540-1550; and Schreiber et al. “Therapeutic Treg expansion in mice by TNFRSF25 prevents allergic lung inflammation” Clin Invest. 2010; 120(10):3629-3640, each of which is incorporated by reference in its entirety.

In embodiments, the second domain is TL1A, wherein the TL1A is capable of binding to a TL1A ligand. In embodiments, the TL1A ligand is DR3. In embodiments, the second domain comprises an amino acid sequence that is at least 95% identical to the amino acid sequence of SEQ ID NO: 70.

In embodiments, where a chimeric protein comprises a variant extracellular domain (ECD) of PD-1, one joining linker preceding an Fc domain, a second joining linker following the Fc domain, and an ECD of TL1A, the chimeric protein may comprise the following structure:
Variant ECD of PD-1-Joining Linker 1-Fc Domain-Joining Linker 2-ECD of TL1A

In embodiments, the extracellular domain of a Type II transmembrane protein is from LIGHT. LIGHT (HVEM-L, TNFSF14, or CD258), an entity homologous to lymphotoxins, with inducible nature, and able to compete with herpes simplex virus glycoprotein D for herpes virus entry mediator (HVEM)/tumor necrosis factor (TNF)-related 2 is a member of the TNF superfamily. It is a 29-kDa Type II transmembrane protein, is expressed as a homotrimer on activated T cells as well as dendritic cells (DCs), and has three receptors, namely, HVEM, LT-β receptor (LTβR, TNFRSF3) and decoy receptor 3 (DcR3). Without wishing to be bound by theory, three receptors with distinct cellular expression patterns have been known to interact with LIGHT: HVEM (TNFRSF14, CD270) detected on activated DCs, T and B cells, natural killer (NK) cells, monocytes, and endothelial cells; LTβR found on follicular DCs and stromal cells and binds LIGHT, and the soluble entity decoy receptor 3 (DcR3) detected on diverse cancer cells such as multiple myeloma and diffuse large B-cell lymphoma. In embodiments, the present variant ECDs of PD-1 and chimeric proteins comprising the variant ECDs can disrupt or decrease the interaction of LIGHT with one or more of these three receptors.

LIGHT binds LTβR, and potentially HVEM as well as DcR3. In embodiments, the present variant ECDs of PD-1 and chimeric proteins comprising the variant ECDs (e.g. comprising the LIGHT ECD) modulate the binding of LIGHT to LTβR (e.g. increase or promote the binding or signal transmission). LTβR is expressed by visceral, lymphoid, and other stroma, epithelia and myeloid cells, but not lymphocytes. In embodiments, the present variant ECDs of PD-1 and chimeric proteins comprising the variant ECDs (e.g. comprising the LIGHT ECD) modulate one or more of visceral, lymphoid, and other stroma, epithelia and myeloid cells. In embodiments, the present variant ECDs of PD-1 and chimeric proteins comprising the variant ECDs (e.g. comprising the LIGHT ECD) modulate the binding of LIGHT to HVEM (e.g. increase or promote the binding or signal transmission). In embodiments, the present variant ECDs of PD-1 and chimeric proteins comprising the variant ECDs (e.g. comprising the LIGHT ECD) modulate the binding of LIGHT to DcR3 (e.g. increase or promote the binding or signal transmission).

In embodiments, the second domain comprises a portion of LIGHT. LIGHT (HVEM-L, TNFSF14, or CD258), an entity homologous to lymphotoxins, with inducible nature, and able to compete with herpes simplex virus glycoprotein D for herpes virus entry mediator (HVEM)/tumor necrosis factor (TNF)-related 2 is a member of the TNF superfamily. LIGHT is a cytokine that binds to that binds to TNFRSF3/LTβR and TNFRSF6B, and TNFRSF14/HVEM. It is a 29-kDa Type II transmembrane protein, is expressed as a homotrimer on activated T cells as well as DCs, and has three receptors, namely, HVEM, LT-β receptor (LTβR, TNFRSF3) and decoy receptor 3 (DcR3). Without wishing to be bound by theory, three receptors with distinct cellular expression patterns have been known to interact with LIGHT: HVEM (TNFRSF14, CD270) detected on activated DCs, T and B cells, NK cells, monocytes, and endothelial cells; LTβR found on follicular DCs and stromal cells and binds LIGHT; and the soluble entity decoy receptor 3 (DcR3) detected on diverse cancer cells such as multiple myeloma and diffuse large B-cell lymphoma. In embodiments, the present variant ECDs of PD-1 and chimeric proteins comprising the variant ECDs can disrupt or decrease the interaction of LIGHT with one or more of these three receptors.

LIGHT binds LTβR, and potentially HVEM as well as DcR3. In embodiments, the present variant ECDs of PD-1 and chimeric proteins comprising the variant ECDs (e.g. comprising the LIGHT ECD) modulate the binding of LIGHT to LTβR (e.g. increase or promote the binding or signal transmission). LTβR is expressed by visceral, lymphoid, and other stroma, epithelia and myeloid cells, but not lymphocytes. In embodiments, the present variant ECDs of PD-1 and chimeric proteins comprising the variant ECDs (e.g. comprising the LIGHT ECD) modulate one or more of visceral, lymphoid, and other stroma, epithelia and myeloid cells. In embodiments, the present variant ECDs of PD-1 and chimeric proteins comprising the variant ECDs (e.g. comprising the LIGHT ECD) modulate the binding of LIGHT to HVEM (e.g. increase or promote the binding or signal transmission). In embodiments, the present variant ECDs of PD-1 and chimeric proteins comprising the variant ECDs (e.g. comprising the LIGHT ECD) modulate the binding of LIGHT to DcR3 (e.g. increase or promote the binding or signal transmission).

In embodiments, the second domain is a portion of LIGHT. In embodiments, the second domain comprises substantially all the extracellular domain of LIGHT. In embodiments, the second domain is capable of binding HVEM, LT-B RECEPTOR (LTβR, TNFRSF3) AND/OR DCR3. In embodiments, the present chimeric protein comprises a domain, e.g., the extracellular domain, of human LIGHT. The human LIGHT comprises the following amino acid sequence:

(SEQ ID NO: 78)
MEESVVRPSVFVVDGQTDIPFTRLGRSHRRQSCSVARVGLGLLLLLMGA
GLAVQGWFLLQLHWRLGEMVTRLPDGPAGSWEQLIQERRSHEVNPAAHL
TGANSSLTGSGGPLLWETQLGLAFLRGLSYHDGALVVTKAGYYYIYSKV
QLGGVGCPLGLASTITHGLYKRTPRYPEELELLVSQQSPCGRATSSSRV
WWDSSFLGGVVHLEAGEKVVVRVLDERLVRLRDGTRSYFGAFMV

The amino acid sequence of extracellular domain human LIGHT (amino acids 50-254 of SEQ ID NO: 78) is the following:

(SEQ ID NO: 71)
LQLHWRLGEMVTRLPDGPAGSWEQLIQERRSHEVNPAAHLTGANSSLTG
SGGPLLWETQLGLAFLRGLSYHDGALVVTKAGYYYIYSKVQLGGVGCPL
GLASTITHGLYKRTPRYPEELELLVSQQSPCGRATSSSRVWWDSSFLGG
VVHLEAGEKVVVRVLDERLVRLRDGTRSYFGAFMV

In embodiments, the present chimeric protein comprises the extracellular domain of human LIGHT which has the amino acid sequence of SEQ ID NO: 71. In embodiments, the present variant ECDs of PD-1 and chimeric proteins comprising the variant ECDs may comprise the extracellular domain of LIGHT as described herein, or a variant or functional fragment thereof. For instance, the chimeric protein may comprise a sequence of the extracellular domain of LIGHT as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of LIGHT as described herein.

OX40L derivatives can be constructed from available structural data, including that described in Mechanistic basis for functional promiscuity in the TNF and TNF receptor superfamilies: structure of the LIGHT:DcR3 assembly, Liu et al., Structure 22: 1252-1262 (2014).

In embodiments, the present variant ECDs of PD-1 and chimeric proteins comprising the variant ECDs may comprise a variant extracellular domain of LIGHT in which the signal peptide (e.g., as provided in SEQ ID NO: 71) is replaced with an alternative signal peptide. In embodiments, the present chimeric protein may comprise a variant extracellular domain of LIGHT which is expressed from a cDNA that has been codon-optimized for expression in protein producing cells such as Chinese Hamster Ovary (CHO) or HEK cells.

In embodiments, the extracellular domain of LIGHT refers to a portion of protein which is capable of interacting with the extracellular environment. In embodiments, the extracellular domain of LIGHT is the entire amino acid sequence of the protein which is external of a cell or the cell membrane. In embodiments, the extracellular domain of LIGHT is a portion of an amino acid sequence of the protein which is external of a cell or the cell membrane and is needed for signal transduction and/or ligand binding as may be assayed using methods know in the art.

In embodiments, the extracellular domain of LIGHT refers to a portion of the protein which is capable for binding to the HVEM, LT-B RECEPTOR (LTβR, TNFRSF3) AND/OR DCR3 receptor. In embodiments, the chimeric protein of the invention binds to human HVEM, LT-B RECEPTOR (LTβR, TNFRSF3) AND/OR DCR3 with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human HVEM, LT-B RECEPTOR (LTBR, TNFRSF3) AND/OR DCR3 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human HVEM, LT-B RECEPTOR (LTBR, TNFRSF3) AND/OR DCR3 with a K_{D} of from about 300 μM to about 700 μM.

In embodiments, the second domain is LIGHT, wherein the LIGHT is capable of binding to a LIGHT receptor. In embodiments, the LIGHT receptor is HVEM, LT-B RECEPTOR (LTβR, TNFRSF3) AND/OR DCR3. In embodiments, the second domain comprises an amino acid sequence that is at least 95% identical to the amino acid sequence of SEQ ID NO: 71.

In embodiments, where a chimeric protein comprises a variant extracellular domain (ECD) of PD-1, one joining linker preceding an Fc domain, a second joining linker following the Fc domain, and an ECD of LIGHT, the chimeric protein may comprise the following structure:
Variant ECD of PD-1-Joining Linker 1-Fc Domain-Joining Linker 2-ECD of LIGHT

In embodiments, chimeric protein is a recombinant chimeric protein.

In embodiments, the linker comprises a polypeptide selected from a flexible amino acid sequence, an IgG hinge region, and an antibody sequence. In embodiments, the linker comprises at least one cysteine residue capable of forming a disulfide bond. In embodiments, the linker comprises an Fc domain. In embodiments, the Fc domain is selected from an IgG Fc domain, an IgA Fc domain, an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the Fc domain comprises hinge-CH2-CH3 Fc domain derived from IgG4. In embodiments, the Fc domain the hinge-CH2-CH3 Fc domain is derived from human IgG4. In embodiments, the Fc domain comprises an amino acid sequence that is at least 95% identical to the amino acid sequence of SEQ ID NO: 1, SEQ ID NO: 2, or SEQ ID NO: 3. In embodiments, the Fc domain comprises hinge-CH2-CH3 Fc domain derived from IgG1. In embodiments, the Fc domain the hinge-CH2-CH3 Fc domain is derived from human IgG1. In embodiments, the chimeric protein further comprises one or more joining linkers, such joining linkers independently selected from SEQ ID NOs: 4-50.

In embodiments, the linker comprises two or more joining linkers each joining linker independently selected from SEQ ID NOs: 4-50; wherein one joining linker is N terminal to the hinge-CH2-CH3 Fc domain and another joining linker is C terminal to the hinge-CH2-CH3 Fc domain.

In embodiments, the chimeric protein or the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present invention, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present invention, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-CH2-CH3 Fc domain and another linker joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an I253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50, or variants thereof are located between an extracellular domain as disclosed herein and an Fc domain as disclosed herein.

In embodiments, the present variant ECDs of PD-1 and chimeric proteins comprising the variant ECDs may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatemers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NOs: 4 to 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTKPR |
| | EEQFNSTYRVVSVLTTPHSDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSRW |
| | QEGNVFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGGGGGSGGGGGGGGSGGGGS |
| 31 | GGGGSGGGGSGGGGSGGGGSGGGGSGGGGSGGGGS |
| 32 | GGGGSGGGGSGGGGSGGGGGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, 5, and E every 4 amino acid intervals.

The combination of a first joining linker, an Fc Domain linker, and a second joining linker is referend to herein as a “modular Iinker”. In embodiments, a chimeric protein comprises a modular linker as shown in Table 2:

TABLE 2
Illustrative modular linkers

| | | | Modular Linker = |
|---|---|---|---|
| Joining Linker | | Joining Linker | Joining Linker 1 + Fc + |
| 1 | Fc | 2 | Joining Linker 2 |
| SKYGPPCPSC | APEFLGGPSVFLFPPKPKDTL | IEGRMD | SKYGPPCPSCPAPEFLGGPSV |
| P | MISRTPEVTCVVVDVSQEDPE | (SEQ ID NO: 7) | FLFPPKPKDTLMISRTPEVTCV |
| (SEQ ID NO: 4) | VQFNWYVDGVEVHNAKTKPR | | WVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: 7) | FLFPPKPKDQLMISRTPEVTCV |
| (SEQ ID NO: 4) | VQFNWYVDGVEVHNAKTKPR | | WVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: 7) | FLFPPKPKDQLMISRTPEVTCV |
| (SEQ ID NO: 4) | VQFNWYVDGVEVHNAKTKPR | | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPC | APEFLGGPSVFLFPPKPKDTL | IEGRMD | SKYGPPCPPCPAPEFLGGPSV |
| P | MISRTPEVTCVVVDVSQEDPE | (SEQ ID NO: 7) | FLFPPKPKDTLMISRTPEVTCV |
| (SEQ ID NO: 5) | VQFNWYVDGVEVHNAKTKPR | | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: 7) | FLFPPKPKDQLMISRTPEVTCV |
| (SEQ ID NO: 5) | VQFNWYVDGVEVHNAKTKPR | | WVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: 7) | FLFPPKPKDQLMISRTPEVTCV |
| (SEQ ID NO: 5) | VQFNWYVDGVEVHNAKTKPR | | WVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the present variant ECDs of PD- and chimeric proteins comprising the variant ECDs may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 51 to 56.

In embodiments, the linker may be flexible, including without limitation highly flexible. In embodiments, the linker may be rigid, including without limitation a rigid alpha helix. Characteristics of illustrative joining linkers are shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR (SEQ ID NO: 16) | Rigid Alpha Helix |
| GGGGSGGGGSGGGGSAS (SEQ ID NO: 17) | Flexible |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP (SEQ ID NO: 23) | Rigid |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

In one aspect, the present disclosure relates to a nucleic acid encoding the chimeric protein of any one of the embodiments disclosed herein. In embodiments, the nucleic acid is an mRNA. In embodiments, the nucleic acid is a DNA.

In one aspect, the present disclosure relates to an expression vector, comprising a nucleic acid encoding the chimeric protein of any of the embodiments disclosed herein.

In one aspect, the present disclosure relates to a host cell, comprising the expression vector of any of the embodiments disclosed herein, mRNA of any of the embodiments disclosed herein, or DNA of any of the embodiments disclosed herein.

In one aspect, the present disclosure relates to a pharmaceutical composition, comprising a therapeutically effective amount of the chimeric protein of any of the embodiments disclosed herein, or the nucleic acid of any of the embodiments disclosed herein, or the expression vector of any of the embodiments disclosed herein, or the host cell of any of the embodiments disclosed herein.

### Diseases; Methods of Treatment, and Patient Selections

In embodiments, the present variant ECDs of PD-1 and chimeric proteins comprising the variant ECDs are capable of, or find use in methods involving, shifting the balance of immune cells in favor of immune attack of a tumor or any other unwanted cells. For instance, the present variant ECDs of PD-1 and chimeric proteins comprising the variant ECDs can shift the ratio of immune cells at a site of clinical importance in favor of cells that can kill a tumor (e.g. T cells, cytotoxic T lymphocytes, T helper cells, natural killer (NK) cells, natural killer T (NKT) cells, anti-tumor macrophages (e.g. M1 macrophages), B cells, and dendritic cells and in opposition to cells that protect tumors (e.g. myeloid-derived suppressor cells (MDSCs), regulatory T cells (Tregs); tumor associated neutrophils (TANs), M2 macrophages, and tumor associated macrophages (TAMs)). In embodiments, the chimeric protein enhances the recognition of tumor antigens by CD8+ T cells and/or enhances tumor infiltration by these T cells.

In aspects, the present chimeric protein of any of the embodiments disclosed herein and/or the recombinant fusion protein of any of the embodiments disclosed herein is used in a method for treating cancer or an inflammatory disease comprising administering an effective amount of a pharmaceutical composition comprising the chimeric protein to a patient in need thereof. In cancer treatment embodiments, for example, the present chimeric protein and/or recombinant fusion protein generates an immune memory response.

Aspects include uses of the present chimeric protein and/or recombinant fusion protein in the manufacture of a medicament, e.g., for treating a cancer and/or an inflammatory disease.

In one aspect, the present disclosure relates to a method of treating cancer or an inflammatory disease, comprising administering an effective amount of a polypeptide comprising one or more variant ECDs of any of the embodiments disclosed herein to a subject in need thereof. In one aspect, the present disclosure relates to a method of treating cancer or an inflammatory disease, comprising administering an effective amount of a composition comprising a polypeptide comprising one or more variant ECDs of any of the embodiments disclosed herein of the present disclosure to a subject in need thereof.

In one aspect, the present disclosure relates to a method of treating cancer or an inflammatory disease, comprising administering an effective amount of a chimeric protein comprising one or more variant ECDs of any of the embodiments disclosed herein to a subject in need thereof. In one aspect, the present disclosure relates to a method of treating cancer or an inflammatory disease, comprising administering an effective amount of a composition comprising a chimeric protein comprising one or more variant ECDs of any of the embodiments disclosed herein of the present disclosure to a subject in need thereof.

In one aspect, the present disclosure relates to a method of treating cancer or an inflammatory disease, comprising administering an effective amount of a pharmaceutical composition of any of the embodiments disclosed herein to a subject in need thereof.

In any of the embodiments disclosed herein, anti-PD-1 antibodies may be present in the sera of the subject. In embodiments, the anti-PD-1 antibodies in sera of subject may be from prior treatments. In embodiments, the anti-PD-1 antibodies in sera of subjects may be from combination treatment with an anti-PD-1 antibody. Accordingly, in embodiments, the subject has received and/or is receiving an anti-PD-1 antibody. In embodiments, the subject has received and/or is receiving an anti-PD-1 antibody selected from pembrolizumab, nivolumab, and cemiplimab.

Further, in embodiments, the present variant ECDs of PD-1 and chimeric proteins comprising the variant ECDs provide synergistic therapeutic effects as it allows for improved site-specific interplay of two immunotherapy agents.

In embodiments, the present variant ECDs of PD-1 and chimeric proteins comprising the variant ECDs provide the potential for reducing off-site and/or systemic toxicity.

In embodiments, the present variant ECDs of PD-1 and chimeric proteins comprising the variant ECDs provide reduced side-effects, e.g., GI complications, relative to current immunotherapies, e.g., antibodies directed to checkpoint molecules as described herein. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

An aspect of the present invention is the use of a herein-disclosed chimeric protein as a medicament in the treatment of a cancer.

Another aspect of the present invention is the use of a herein-disclosed chimeric protein, in the manufacture of a medicament.

Yet another aspect of the present invention is an expression vector comprising a nucleic acid that encodes a herein-disclosed chimeric protein.

In an aspect, the present invention provides a host cell comprising an expression vector that comprises a nucleic acid that encodes a herein-disclosed chimeric protein.

In embodiments, the present invention pertains to cancers and/or tumors; for example, the treatment or prevention of cancers and/or tumors. As described elsewhere herein, the treatment of cancer may involve In embodiments, modulating the immune system with the present variant ECDs of PD-1 and chimeric proteins comprising the variant ECDs to favor immune stimulation over immune inhibition.

In one aspect, the present disclosure relates to a method of treating cancer or an inflammatory disease, comprising administering an effective amount of a pharmaceutical composition of any of the embodiments disclosed herein to a subject in need thereof.

In one aspect, the present disclosure relates to a method of modulating a patient's immune response, comprising administering an effective amount of a pharmaceutical composition of any of the embodiments disclosed herein to a subject in need thereof.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g. virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis.

The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present invention include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the chimeric protein is used to treat a subject that has a treatment-refractory cancer. In embodiments, the chimeric protein is used to treat a subject that is refractory to one or more immune-modulating agents. For example, in embodiments, the chimeric protein is used to treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g. ipilimumab (YERVOY)-refractory patients (e.g. melanoma patients). Accordingly, in embodiments the present invention provides methods of cancer treatment that rescue patients that are non-responsive to various therapies, including monotherapy of one or more immune-modulating agents.

In embodiments, the present methods provide treatment with the chimeric protein in a patient who is refractory to an additional agent, such “additional agents” being described elsewhere herein, inclusive, without limitation, of the various chemotherapeutic agents described herein.

In embodiments, the chimeric proteins are used to treat, control or prevent one or more inflammatory diseases or conditions. Non-limiting examples of inflammatory diseases include acne vulgaris, acute inflammation, allergic rhinitis, asthma, atherosclerosis, atopic dermatitis, autoimmune disease, autoinflammatory diseases, autosomal recessive spastic ataxia, bronchiectasis, celiac disease, chronic cholecystitis, chronic inflammation, chronic prostatitis, colitis, diverticulitis, familial eosinophilia (FE), glomerulonephritis, glycerol kinase deficiency, hidradenitis suppurativa, hypersensitivities, inflammation, inflammatory bowel diseases, inflammatory pelvic disease, interstitial cystitis, laryngeal inflammatory disease, Leigh syndrome, lichen planus, mast cell activation syndrome, mastocytosis, ocular inflammatory disease, otitis, pain, pelvic inflammatory disease, reperfusion injury, respiratory disease, restenosis, rheumatic fever, rheumatoid arthritis, rhinitis, sarcoidosis, septic shock, silicosis and other pneumoconioses, transplant rejection, tuberculosis, and vasculitis.

In various embodiments, the inflammatory disease is an autoimmune disease or condition, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In aspects, the present chimeric agents are used in methods of activating a T cell, e.g. via the extracellular domain of 4-1BBL or CD30L.

In aspects, the present chimeric agents are used in methods of preventing the cellular transmission of an immunosuppressive signal via the extracellular domain of variant PD-1.

### Formulations

The chimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In one aspect, the present disclosure relates to a pharmaceutical composition, comprising a therapeutically effective amount of the recombinant protein of any of the embodiments disclosed herein, chimeric protein of any of the embodiments disclosed herein, the heterologous chimeric protein of any of the embodiments disclosed herein, or the nucleic acid of any of the embodiments disclosed herein, or the expression vector of any of the embodiments disclosed herein, or the host cell of any of the embodiments disclosed herein.

In embodiments, the compositions described herein are in the form of a pharmaceutically acceptable salt.

Further, any chimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are suspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

### Administration, Dosing, and Treatment Regimens

The present invention includes the described chimeric protein (and/or additional agents) in various formulations. Any chimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the chimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the chimeric protein (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art) In embodiments, any chimeric protein (and/or additional agents) described herein is formulated in accordance with routine procedures as a composition adapted for a mode of administration described herein.

Routes of administration include, for example: intradermal, intratumoral, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In some instances, administration results in the release of any agent described herein into the bloodstream, or alternatively, the agent is administered directly to the site of active disease.

Any chimeric protein (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In embodiments, for instance in the treatment of cancer, the chimeric protein (and/or additional agents) are administered in the tumor microenvironment (e.g. cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the chimeric protein (and/or additional agents) are administered intratumorally.

In embodiments, the present chimeric protein allows for a dual effect that provides less side effects than are seen in conventional immunotherapy (e.g. treatments with one or more of OPDIVO, KEYTRUDA, YERVOY, and TECENTRIQ). For example, the present variant ECDs of PD-1 and chimeric proteins comprising the variant ECDs reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g. intratumorally, obviate adverse event seen with standard systemic administration, e.g. IV infusions, as are used with conventional immunotherapy (e.g. treatments with one or more of OPDIVO, KEYTRUDA, YERVOY, and TECENTRIQ).

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

The dosage of any chimeric protein (and/or additional agents) described herein as well as the dosing schedule can depend on various parameters, including, but not limited to, the disease being treated, the subject's general health, and the administering physician's discretion. Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart.

In embodiments, the present invention relates to the co-administration of the present chimeric protein comprising the extracellular domain of variant PD-1 and another chimeric protein which induces an innate immune response. In such embodiments, the present chimeric protein may be administered before, concurrently with, or subsequent to administration of the chimeric protein which induces an innate immune response. For example, the chimeric proteins may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart. In an exemplary embodiment, the present chimeric protein comprising the extracellular domain of variant PD-1 and the chimeric protein which induces an innate immune response are administered 1 week apart, or administered on alternate weeks (i.e., administration of the chimeric protein inducing an innate immune response is followed 1 week later with administration of the present chimeric protein comprising the extracellular domain of variant PD-1 and so forth).

The dosage of any chimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In embodiments, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any chimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or additional agents) described herein can be administered in a single daily dose, or the total daily dosage can be administered in divided doses of two, three or four times daily. Furthermore, any chimeric protein (and/or additional agents) described herein can be administered continuously rather than intermittently throughout the dosage regimen.

### Cells and Nucleic Acids

In embodiments, the present invention provides an expression vector, comprising a nucleic acid encoding the chimeric protein described herein. In embodiments, the expression vector comprises DNA or RNA. In embodiments, the expression vector is a mammalian expression vector.

In one aspect, the present disclosure relates to a nucleic acid encoding the recombinant protein of any of the embodiments disclosed herein, chimeric protein of any of the embodiments disclosed herein, or the heterologous chimeric protein of any of the embodiments disclosed herein. In embodiments, the nucleic acid is an mRNA. In embodiments, the nucleic acid is a DNA.

In one aspect, the present disclosure relates to an expression vector, comprising a nucleic acid encoding the heterologous chimeric protein of any of the embodiments disclosed herein.

In one aspect, the present disclosure relates to a host cell, comprising the expression vector of any of the embodiments disclosed herein, mRNA of any of the embodiments disclosed herein, or DNA of any of the embodiments disclosed herein.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful.

A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the invention comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In embodiments, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present invention contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used fortargeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric proteins including CRISPR/CAS9, zinc finger, TALEN, and meganuclease gene-editing technologies.

In one aspect, the invention provides expression vectors for the expression of the chimeric proteins (and/or additional agents) that are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and α viruses, though other viral vectors may also be used. For in vivo uses, viral vectors that do not integrate into the host genome are suitable for use, such as a viruses and adenoviruses. Illustrative types of α viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

In embodiments, the present invention provides a host cell, comprising the expression vector comprising the chimeric protein described herein.

Expression vectors can be introduced into host cells for producing the present variant ECDs of PD-1 and chimeric proteins comprising the variant ECDs. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present variant ECDs of PD-1 and chimeric proteins comprising the variant ECDs in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g. GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In embodiments, the patient has received and/or is receiving a treatment with an anti-PD-1 agent such as an anti-PD-1 antibody. In embodiments, the patient has received and/or is receiving pembrolizumab (e.g., KEYTRUDA). In embodiments, the patient has received and/or is receiving nivolumab (e.g., OPDIVO). In embodiments, the patient has received and/or is receiving cemiplimab (LIBTAYO). In embodiments, the patient has received and/or is receiving an anti-PD-1 antibody selected from pembrolizumab, nivolumab, and cemiplimab. Accordingly, in embodiments, anti-PD-1 antibodies may be present in the sera of the patient. In embodiments, the anti-PD-1 antibody present in the sera of the patient may be from a prior treatment with an anti-PD-1 antibody (e.g., pembrolizumab, nivolumab, and cemiplimab). In embodiments, the anti-PD-1 antibody present in the sera of the patient may be from combination treatment with an anti-PD-1 antibody (e.g., pembrolizumab, nivolumab, and cemiplimab).

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use.

In a specific embodiment, the non-human animal is a household pet. In another specific embodiment, the non-human animal is a livestock animal.

### Kits

The invention provides kits that can simplify the administration of any agent described herein. An illustrative kit of the invention comprises any composition described herein in unit dosage form. In embodiments, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In embodiments, the kit comprises a container containing an effective amount of a composition of the invention and an effective amount of another composition, such those described herein.

Any aspect or embodiment described herein can be combined with any other aspect or embodiment as disclosed herein.

The invention will be further described in the following examples, which do not limit the scope of the invention described in the claims.

### EXAMPLES

The examples herein are provided to illustrate advantages and benefits of the present disclosure and to further assist a person of ordinary skill in the art with preparing anti-PD-1 agents that are not neutralized by anti-PD-1 antibodies. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Creation of PD-1-ECD Mutations at R86 and/or Q88

Nivolumab and pembrolizumab are believed to cause the blockade of the PD-1-PD-L1/2 interaction. In efforts towards making variant ECD of PD-1, that retain binding to PD-1/2, while reducing binding to pembrolizumab, amino acid residues R86 and Q88 of PD-1 were altered to create R86A and/or Q88E substitution derivatives of PD-1. These mutations were designed to change local charge and were likely to alter local electrostatic interactions, if any, between PD-1 and pembrolizumab. PD-1-Fc-4-1BBL chimeric protein (see WO2017059168, the entire contents of which are hereby incorporated by reference) harboring these mutations were created. These chimeric fusion proteins were called the PD-1(R86A)-Fc-4-1BBL, PD-1(Q88E)-Fc-4-1BBL, and PD-1(R86A-Q88E)-Fc-4-1BBL chimeric proteins.

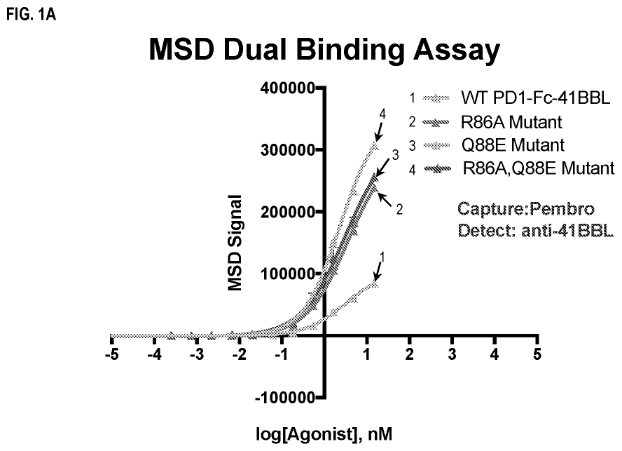

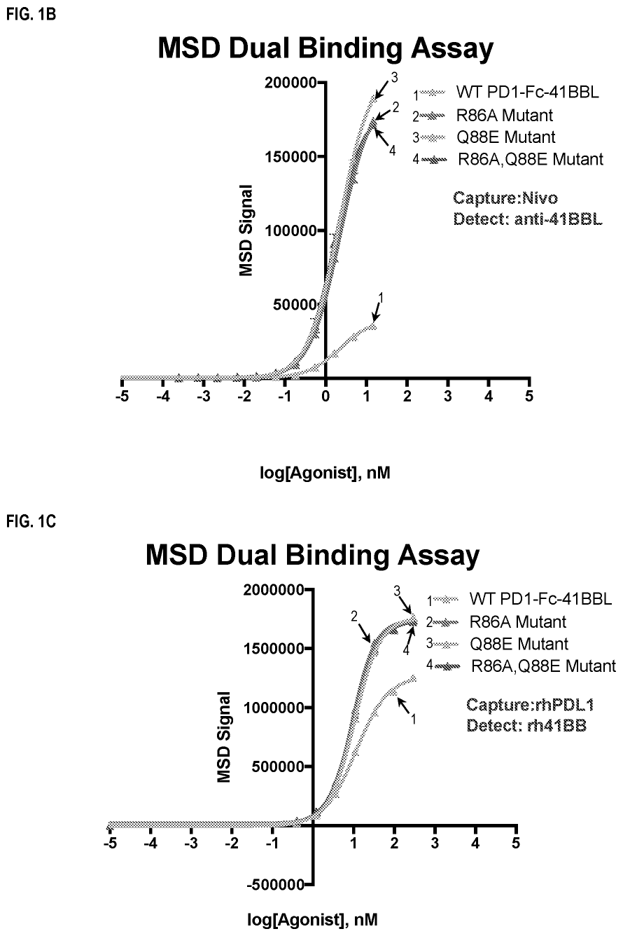

These fusion chimeric protein mutants were purified and their binding to pembrolizumab was evaluated using the Meso Scale Discovery (MSD) ELISA assays. Toward that, pembrolizumab was coated on plates and increasing amounts of the PD-1-Fc-4-1BBL, PD-1(R86A)-Fc-4-1BBL, PD-1(Q88E)-Fc-4-1BBL, and PD-1(R86A-Q88E)-Fc-4-1BBL chimeric proteins were added to the plates for capture by the plate-bound pembrolizumab. Binding of the chimeric proteins to pembrolizumab was detected using an anti-4-1BBL antibody, and using an electrochemiluminescence (ECL) readout. As shown in FIG. 1A, each of the PD-1(R86A)-Fc-4-1BBL, PD-1(Q88E)-Fc-4-1BBL, and PD-1(R86A-Q88E)-Fc-4-1BBL chimeric proteins bound to pembrolizumab better compared to the PD-1-Fc-4-1BBL chimeric protein. These data indicate that electrostatic interactions involving R86 and/or Q88, if any, do not play a significant role in binding of pembrolizumab to PD-1.

The binding of these proteins to nivolumab was then evaluated. Nivolumab was coated on plates and increasing amounts of the PD-1-Fc-4-1BBL, PD-1(R86A)-Fc-4-1BBL, PD-1(Q88E)-Fc-4-1BBL, and PD-1(R86A-Q88E)-Fc-4-1BBL chimeric proteins were added to the plates for capture by the plate-bound recombinant nivolumab. Binding of was nivolumab to the chimeric proteins was detected using an anti-4-1BBL antibody, and using an electrochemiluminescence (ECL) readout. As shown in FIG. 1B, each of the PD-1(R86A)-Fc-4-1BBL, PD-1(Q88E)-Fc-4-1BBL, and PD-1(R86A-Q88E)-Fc-4-1BBL chimeric proteins bound also to nivolumab better than that of the PD-1-Fc-4-1BBL chimeric protein.

Then, the binding of these proteins to PD-L1 was evaluated. Recombinant human PD-L1 (rhPD-L1) was coated on plates and increasing amounts of the PD-1-Fc-4-1BBL, PD-1(R86A)-Fc-4-1BBL, PD-1(Q88E)-Fc-4-1BBL, and PD-1(R86A-Q88E)-Fc-4-1BBL chimeric proteins were added to the plates for capture by the plate-bound recombinant rhPD-L1. The binding was detected using recombinant human 4-1BB (rh4-1BB) protein using an electrochemiluminescence (ECL) readout. As shown in FIG. 1C, each of the PD-1(R86A)-Fc-4-1BBL, PD-1(Q88E)-Fc-4-1BBL, and PD-1(R86A-Q88E)-Fc-4-1BBL chimeric proteins bound to rhPD-L1 better compared to the PD-1-Fc-4-1BBL chimeric protein.

These data indicate that the R86A and/or Q88E mutations likely stabilize a part of PD-1 molecule, allowing more efficient binding of each of pembrolizumab, nivolumab, and PD-L1. However, these data validate the strategy of rational mutagenesis for altering the affinity of PD-1 to its ligands.

### Example 2: Creation of PD-1-ECD Mutations

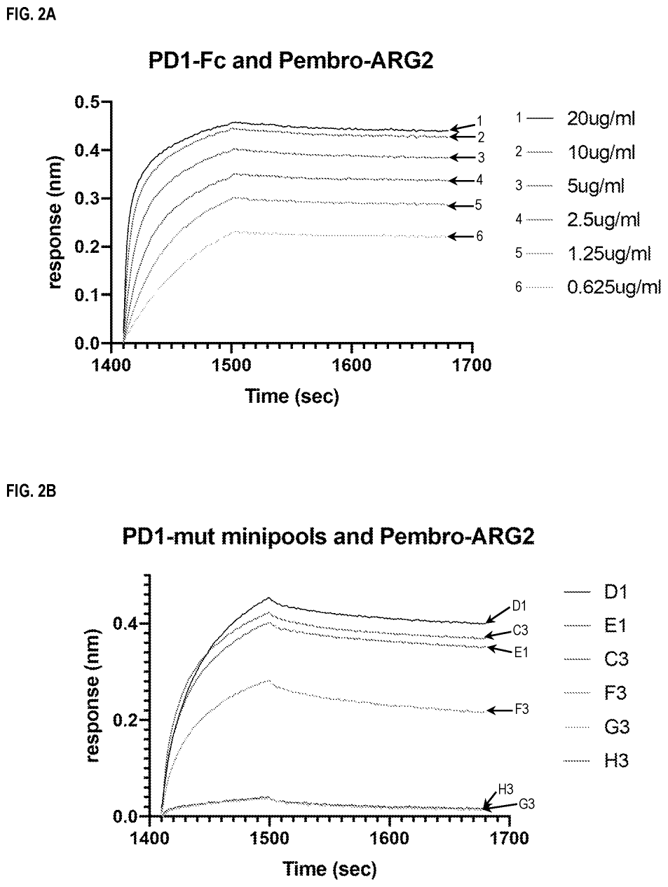

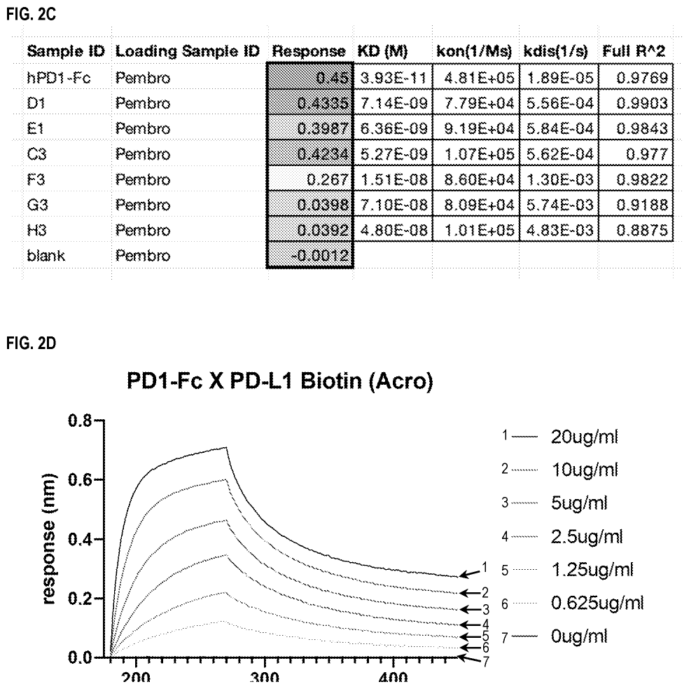

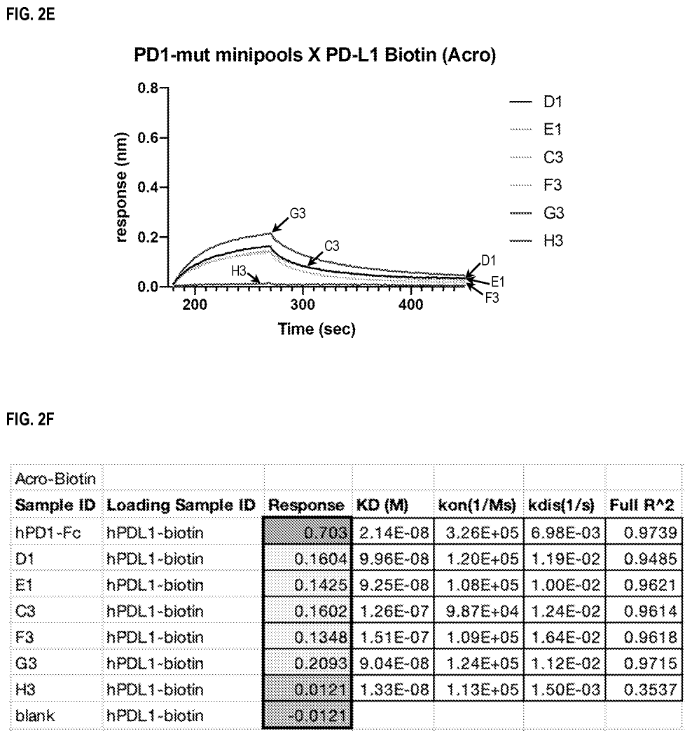

Further amino acid residues including S87 (FIGS. 1B and 2A) of PD-1 were altered to create a series of mutants designated as D1, E1, C3, F3, and G3 and wild type or mutant derivatives of the PD-1-Fc fusion protein were generated. These fusion protein mutants were purified and their binding to pembrolizumab and PD-L1 as measured by bio-layer interferometry using the Octet system (ForteBio) in comparison to the PD-1-Fc fusion protein.

In one experiment, wild-type PD-1-Fc fusion protein was used as a reference to characterize the binding to pembrolizumab. Briefly, pembrolizumab was immobilized on a biosensor tip. The biosensor tip was dipped into solutions containing increasing concentrations of the PD-1-Fc fusion protein and binding kinetics was measured with bio-layer interferometry. As shown in FIG. 2A, PD-1-Fc fusion protein exhibited increasing binding to pembrolizumab with increasing concentrations of the PD-1-Fc fusion protein. The binding kinetics of the mutant PD-1-Fc fusion protein derivatives D1, E1, C3, F3, and G3 to pembrolizumab was then studied using bio-layer interferometry. A commercially available PD-1-Fc fusion protein (designated as H3) was also included. Briefly, pembrolizumab was immobilized on a biosensor tip. The biosensor tip was dipped into solutions containing the indicated mutant protein and binding kinetics was measured. As shown in FIG. 2B, G3 (the PD-1-Fc fusion protein having S87C substitution (the PD-1(S87C)-Fc fusion protein)) showed significantly reduced binding to pembrolizumab compared to the PD-1-Fc fusion protein (see also FIG. 2C). D1, E1 and C3 showed efficient binding and F3 showed slightly reduced binding to pembrolizumab (FIG. 2B). H3, the commercially available PD-1-Fc fusion protein, showed no activity in this assay possibly suggesting the protein may be inactivated. FIG. 2C shows the kinetic parameters (kₒₙ, k_{dis}, K_{D} and R²) of the binding of the PD-1-Fc fusion protein or the D1, E1, C3, F3, and the PD-1(S87C)-Fc mutant fusion proteins to pembrolizumab. As shown in FIG. 2C, the PD-1(S87C)-Fc fusion protein exhibited a decrease in kₒₙ, k_{dis} as well as K_{D}, compared to PD-1-Fc fusion protein.

The binding of the D1, E1, C3, F3, and the PD-1(S87C)-Fc mutant fusion proteins to PD-L1 protein was also studied in comparison with the binding of the PD-1-Fc fusion protein bio-layer Interferometry using the Octet system (ForteBio). To characterize the binding the wild-type PD-1-Fc fusion protein, which was used as a reference, PD-L1 protein was immobilized on a biosensor tip. The biosensor tip was dipped into solutions containing increasing concentrations of the PD-1-Fc fusion protein and binding kinetics was measured. As shown in FIG. 2D, the PD-1-Fc fusion protein bound to PD-L1 in a concentration-dependent manner. Then, the binding kinetics to PD-L1 protein by the mutant PD-1-Fc fusion protein derivatives D1, E1, C3, F3, and the PD-1(S87C)-Fc mutant fusion was studied using bio-layer interferometry. The commercially available PD-1-Fc fusion protein (designated as H3) was also included. Briefly, PD-L1 protein was immobilized on a biosensor tip. The biosensor tip was dipped into solutions containing the indicated mutant protein and binding kinetics was measured. As shown in FIG. 2E, each of the mutant PD-1-Fc fusion protein exhibited decreased binding to PD-L1 compared to the PD-1-Fc fusion protein. Interestingly, as shown in FIG. 2E, the PD-1(S87C)-Fc fusion protein (G3) showed significant binding to PD-L1 compared to the PD-1-Fc fusion protein (FIG. 2B). H3, the commercially available PD-1-Fc fusion protein, showed no activity in this assay possibly suggesting the protein may be inactivated. FIG. 2F shows the kinetic parameters (kₒₙ, k_{dis}, K_{D} and R²) for the binding of the PD-1-Fc fusion protein or the mutants thereof to PD-L1.

These data demonstrate that the PD-1(S87C)-Fc mutant fusion protein showed significantly reduced binding to pembrolizumab compared to the PD-1-Fc fusion protein, while retaining the ability to bind PD-L1.

### Example 3: Additional Mutations of the S87 and its Neighboring Amino Acid Residues of PD-1

Next amino acid 87 was focused upon, and additional created other single mutants of S87, as well as multi-mutants of S87 and neighboring amino acids were constructed. Specifically, the following mutant fusion proteins were created: PD-1(S87K)-Fc, PD-1(S87D)-Fc, PD-1(S87F)-Fc, PD-1(S87A)-Fc, PD-1(R86A-S87C-Q88E)-Fc, PD-1(R86A-S87C)-Fc, and PD-1(S87C-Q88E)-Fc.

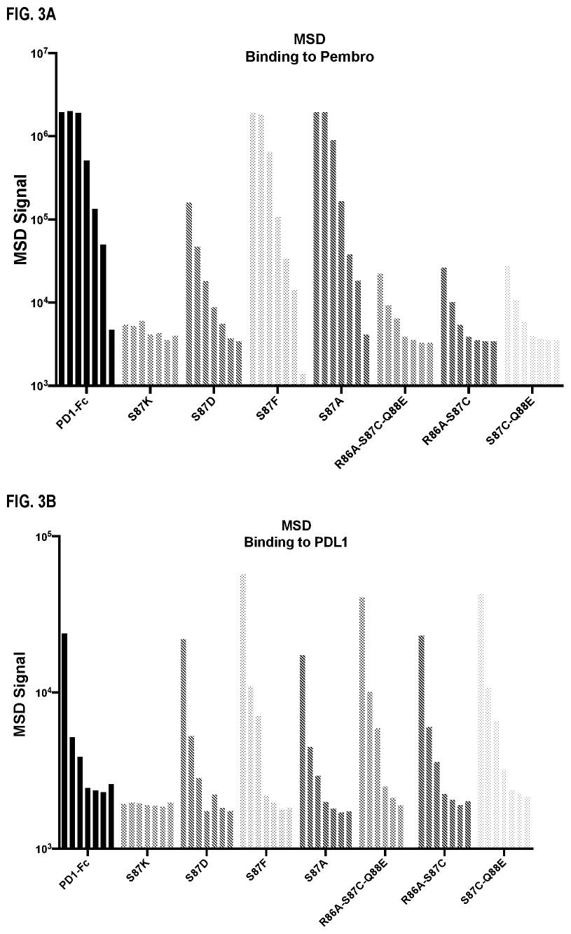

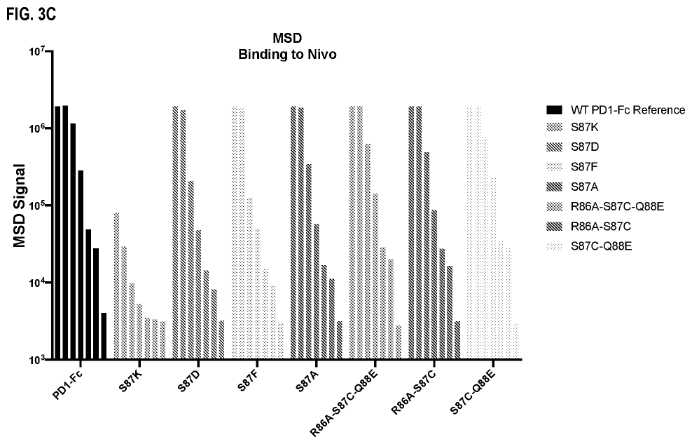

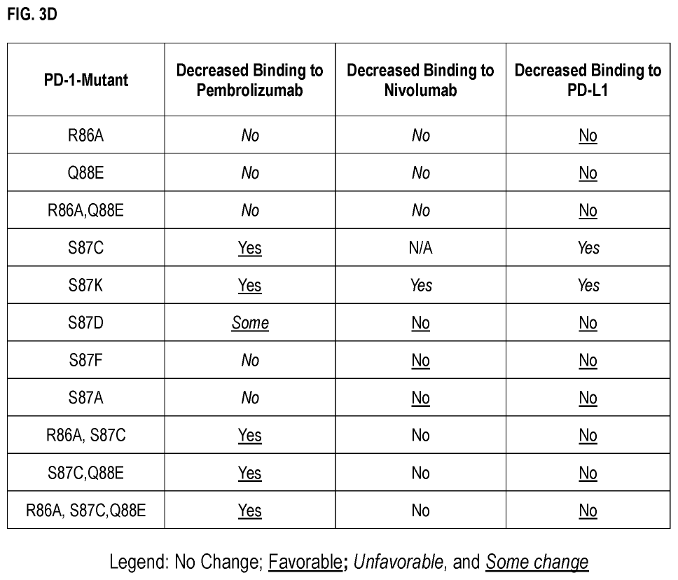

These fusion protein mutants were purified and their binding to pembrolizumab, nivolumab and PD-L1 as measured in comparison to the PD-1-Fc fusion protein using the Meso Scale Discovery (MSD) ELISA assays. Briefly, pembrolizumab was coated on plates and incubated with increasing concentrations (10, 3.33, 1.11, 0.37, 0.12, 0.04 or 0 μg/ml) of the PD-1-Fc fusion protein or the above mutants (S87K, S87D, S87F, S87A, R86A-S87C-Q88E, R86A-S87C, and S87C-Q88E) for capture by the plate-bound pembrolizumab. The binding was detected using an anti-Fc antibody using an electrochemiluminescence (ECL) readout. MDS signal was plotted was a function of concentration of the given fusion protein. As shown in FIG. 3A, the PD-1(S87K)-Fc, PD-1(R86A-S87C-Q88E)-Fc, PD-1(R86A-S87C)-Fc, or PD-1(S87C-Q88E)-Fc proteins exhibited decreased binding to pembrolizumab compared to the PD-1-Fc fusion protein. The binding of the PD-1(S87D)-Fc fusion protein was slightly decreased compared to that of the PD-1-Fc fusion protein (FIG. 3A). The extent of binding of the PD-1(S87F)-Fc and PD-1(S87A)-Fc fusion proteins was similar to that of the PD-1-Fc fusion protein (FIG. 3A).

To measure the binding of the fusion proteins to PD-L1, briefly, PD-L1 was coated on plates and incubated with increasing concentrations (10, 3.33, 1.11, 0.37, 0.12, 0.04 or 0 μg/ml) of the PD-1-Fc fusion protein or mutants thereof for capture by the plate-bound recombinant PD-L1. The mutants that were tested were the following: PD-1(S87K)-Fc, PD-1(S87D)-Fc, PD-1(S87F)-Fc, PD-1(S87A)-Fc, PD-1(R86A-S87C-Q88E)-Fc, PD-1(R86A-S87C)-Fc, or PD-1(S87C-Q88E)-Fc. The binding was detected using an anti-Fc antibody using an electrochemiluminescence (ECL) readout. MDS signal was plotted was a function of concentration of the given fusion protein. As shown in FIG. 3B, the binding of the PD-1(S87D)-Fc, PD-1(S87F)-Fc and PD-1(S87A)-Fc, PD-1(R86A-S87C-Q88E)-Fc, PD-1(R86A-S87C)-Fc, or PD-1(S87C-Q88E)-Fc fusion proteins was not significantly affected compared to the PD-1-Fc fusion protein. The PD-1(S87K)-Fc fusion protein exhibited decreased binding to PD-L1 compared to the PD-1-Fc fusion protein. (FIG. 3B).

The binding of the fusion proteins to nivolumab was then measured in comparison to the PD-1-Fc fusion protein using MSD ELISA assays. Briefly, nivolumab was coated on plates and incubated with increasing concentrations (10, 3.33, 1.11, 0.37, 0.12, 0.04 or 0 μg/ml) of the PD-1-Fc fusion protein or mutants thereof for capture by the plate-bound recombinant nivolumab. The mutants that were tested were the following: PD-1(S87K)-Fc, PD-1(S87D)-Fc, PD-1(S87F)-Fc, PD-1(S87A)-Fc, PD-1(R86A-S87C-Q88E)-Fc, PD-1(R86A-S87C)-Fc, or PD-1(S87C-Q88E)-Fc. The binding was detected using an anti-Fc antibody using an electrochemiluminescence (ECL) readout. As shown in FIG. 3C, the PD-1(S87K)-Fc fusion protein exhibited decreased binding to nivolumab compared to the PD-1-Fc fusion protein. The PD-1(S87D)-Fc, PD-1(S87F)-Fc, PD-1(S87A)-Fc, PD-1(R86A-S87C-Q88E)-Fc, PD-1(R86A-S87C)-Fc, or PD-1(S87C-Q88E)-Fc fusion proteins bound nivolumab similar to the PD-1-Fc fusion protein (FIG. 3C). FIG. 3D shows summary of binding of the mutants disclosed herein. As shown in FIG. 3D, the R86A, S87C; S87C,Q88E; and R86A, S87C,Q88E mutants of PD-1 showed decreased biding to pembrolizumab but not to PD-L1.

### Example 4: Binding of the PD-1-Fc-OX40L Chimeric Fusion Protein or its Mutant Derivatives to PD-L1 Pembrolizumab, or Nivolumab

The PD-1(S87D)-Fc-OX40L, PD-1(R86A-S87C)-Fc-OX40L, PD-1(R86A-S87C-Q88E)-Fc-OX40L chimeric fusion proteins were generated and their ability to bind pembrolizumab, PD-L1, and nivolumab, PD-L1, and nivolumab was compared with the PD-1-Fc-OX40L chimeric protein (comprising the extracellular domain of wild type PD-1) using Meso Scale Discovery (MSD) ELISA assays.

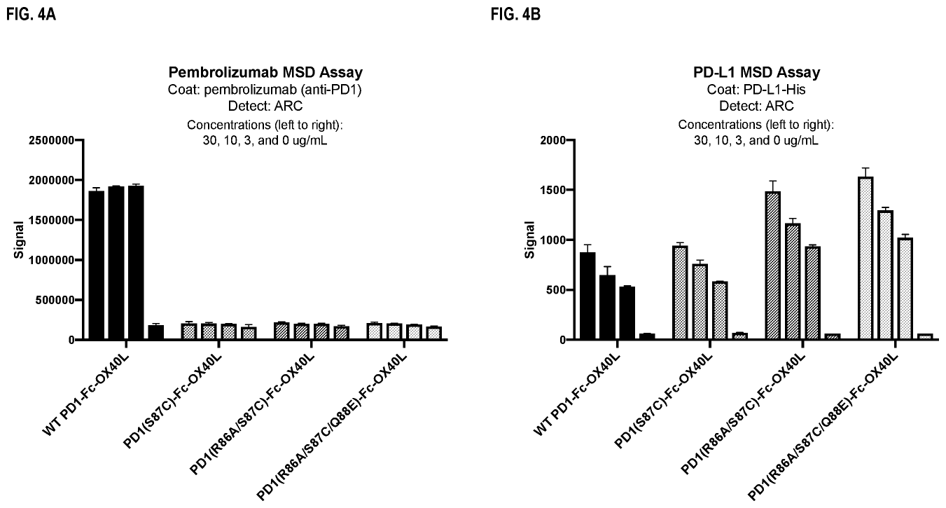

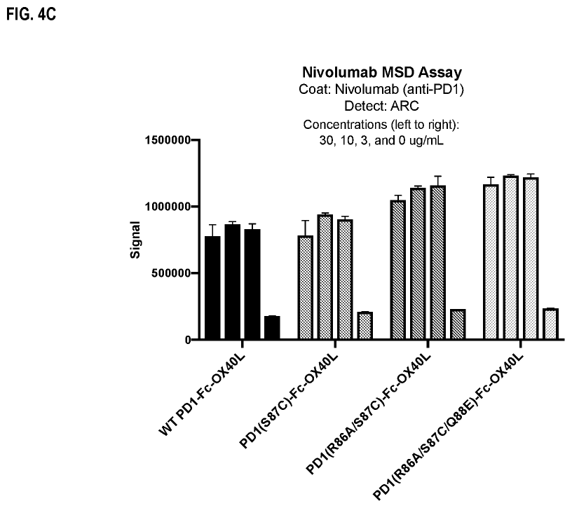

The binding of the PD-1-Fc-OX40L chimeric fusion protein or mutants thereof to pembrolizumab was measured using MSD ELISA assays. Briefly, pembrolizumab was coated on plates and incubated with decreasing concentrations (30, 10, 3, 1, or 0 μg/ml) of the PD-1-Fc-OX40L, PD-1(S87D)-Fc-OX40L, PD-1(R86A-S87C)-Fc-OX40L, PD-1(R86A-S87C-Q88E)-Fc-OX40L chimeric proteins for capture by the plate-bound pembrolizumab. The binding was detected using an anti-Fc antibody using an electrochemiluminescence (ECL) readout. As shown in FIG. 4A, each of the −1(S87D)-Fc-OX40L, PD-1(R86A-S87C)-Fc-OX40L, PD-1(R86A-S87C-Q88E)-Fc-OX40L chimeric fusion proteins showed only a background signal. In contrast, the PD-1-Fc-OX40L chimeric fusion protein exhibited binding to pembrolizumab (FIG. 4A). These results indicate that the chimeric proteins comprising the mutant PD-1 protein disclosed herein do not bind pembrolizumab.

The binding of the PD-1-Fc-OX40L chimeric fusion protein or mutants thereof to recombinant PD-L1-His was measured using MSD ELISA assays. Briefly, recombinant PD-L1-His was coated on plates and incubated with decreasing concentrations (30, 10, 3, 1, or 0 μg/ml) of the PD-1-Fc-OX40L, PD-1(S87D)-Fc-OX40L, PD-1(R86A-S87C)-Fc-OX40L, PD-1(R86A-S87C-Q88E)-Fc-OX40L chimeric fusion proteins for capture by the plate-bound recombinant PD-L1-His. The binding was detected using an anti-Fc antibody using an electrochemiluminescence (ECL) readout. As shown in FIG. 4B, the extent of binding of the PD-1(S87D)-Fc-OX40L, PD-1(R86A-S87C)-Fc-OX40L, PD-1(R86A-S87C-Q88E)-Fc-OX40L chimeric fusion proteins to recombinant PD-L1-His was similar to the PD-1-Fc-OX40L chimeric fusion protein. These results indicate that the chimeric proteins comprising the mutant PD-1 protein disclosed herein can bind to PD-L1 protein.

The binding of the PD-1-Fc-OX40L chimeric fusion protein or mutants thereof to nivolumab was then measured using MSD ELISA assays. Briefly, nivolumab was coated on plates and incubated with decreasing concentrations (30, 10, 3, 1, or 0 μg/ml) of the PD-1-Fc-OX40L, PD-1(S87D)-Fc-OX40L, PD-1(R86A-S87C)-Fc-OX40L, PD-1(R86A-S87C-Q88E)-Fc-OX40L chimeric fusion proteins for capture by the plate-bound nivolumab. The binding was detected using an anti-Fc antibody using an electrochemiluminescence (ECL) readout. As shown in FIG. 4C, the binding of the PD-1(S87D)-Fc-OX40L, PD-1(R86A-S87C)-Fc-OX40L, PD-1(R86A-S87C-Q88E)-Fc-OX40L chimeric fusion proteins to nivolumab was similar to the PD-1-Fc-OX40L chimeric fusion protein. These results indicate that the chimeric proteins comprising the mutant PD-1 protein disclosed herein can bind to nivolumab.

Collectively, these results indicate that the chimeric proteins comprising the mutant PD-1 protein disclosed herein bind to PD-L1 (or nivolumab) but not to pembrolizumab.

### Example 5: Inhibition by Pembrolizumab of Binding of the PD-1-Fc-OX40L Chimeric Fusion Protein and its Mutant Derivatives to Cells Expressing PD-L1

The effect of pembrolizumab on binding of the PD-1-Fc-OX40L, PD-1(S87D)-Fc-OX40L, PD-1(R86A-S87C)-Fc-OX40L, or PD-1(R86A-S87C-Q88E)-Fc-OX40L chimeric proteins to cells expressing PD-L1 was studied. For this purpose, CHO-K1 cells expressing human PD-L1 (the CHO-K1/hPD-L1 cells) were used. Fromm et al., Agonist redirected checkpoint, for cancer immunotherapy, J Immunother Cancer 6(1):149 (2018).

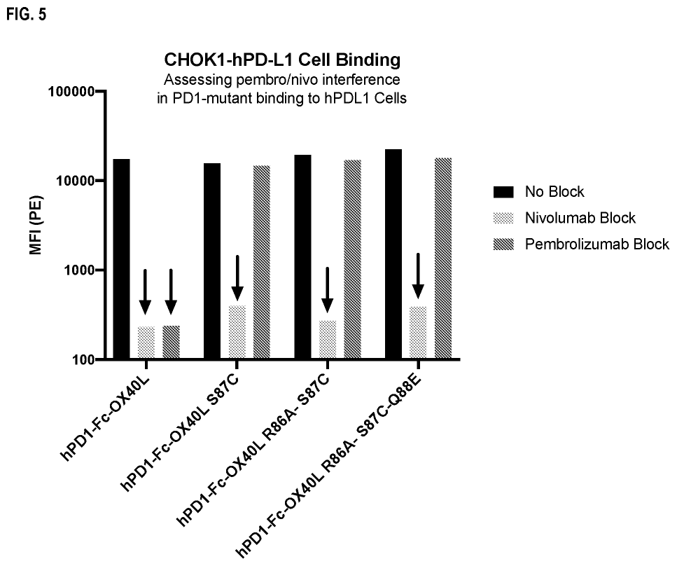

20 μg/mL of the PD-1-Fc-OX40L, PD-1(S87D)-Fc-OX40L, PD-1(R86A-S87C)-Fc-OX40L, or PD-1(R86A-S87C-Q88E)-Fc-OX40L chimeric proteins were pre-incubated with buffer alone, 50 μg/mL nivolumab, or 50 μg/mL pembrolizumab for 1 hour at room temperature. Following this incubation, the mixture was added to the CHO-K1/hPD-L1 cells and incubated for 30 minutes on ice. The CHO-K1/hPD-L1 cells were then washed once in PBS and an antibody specific to the Fc domain present in the PD-1-Fc-OX40L chimeric fusion protein or mutants thereof was used to detect binding to the cells. The binding was assessed by flow cytometry. Mean fluorescence intensity (MFI) was plotted as a function of the treatment and plotted. The MFI for each of the PD-1(S87D)-Fc-OX40L, PD-1(R86A-S87C)-Fc-OX40L, or PD-1(R86A-S87C-Q88E)-Fc-OX40L chimeric proteins that were pretreated with buffer only (no block) was comparable to that of the PD-1-Fc-OX40L chimeric protein (FIG. 5), indicating that the PD-1(S87D)-Fc-OX40L, PD-1(R86A-S87C)-Fc-OX40L, or PD-1(R86A-S87C-Q88E)-Fc-OX40L chimeric proteins bound PD-L1-expressing cells as efficiently as the PD-1-Fc-OX40L chimeric protein. As expected, both nivolumab and pembrolizumab reduced the MFI for the binding of the PD-1-Fc-OX40L protein, indicating a blockade of binding to PD-L1-expressing cells by nivolumab and pembrolizumab (FIG. 5). Interestingly, as shown in FIG. 5, preincubation with pembrolizumab had no effect on binding of the PD-1(S87D)-Fc-OX40L, PD-1(R86A-S87C)-Fc-OX40L, or PD-1(R86A-S87C-Q88E)-Fc-OX40L chimeric proteins to the CHO-K1/hPD-L1 cells. On the other hand, preincubation with nivolumab blocked the binding of the PD-1(S87D)-Fc-OX40L, PD-1(R86A-S87C)-Fc-OX40L, or PD-1(R86A-S87C-Q88E)-Fc-OX40L chimeric proteins to the CHO-K1/hPD-L1 cells.

These results indicate that the PD-1(S87D)-PD-1(R86A-S87C) and PD-1(R86A-S87C-Q88E) mutations eliminated the blocking effect of pembrolizumab, but not nivolumab, on the binding to the CHO-K1/hPD-L1 cells.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A polypeptide comprising a variant extracellular domain (ECD) of PD-1, wherein the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57, wherein the variant ECD and/or the polypeptide has less affinity to pembrolizumab and/or nivolumab compared to a wild type PD-1 ECD having an amino acid sequence that is identical to the amino acid sequence of SEQ ID NO: 58.

2. The polypeptide of claim 1, wherein the variant ECD and/or the polypeptide has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58.

3. The polypeptide of claim 1 or claim 2, wherein the variant ECD and/or the polypeptide has an affinity to a PD-1 ligand that is equivalent to the affinity of the wild type PD-1 ECD having an amino acid sequence that is identical to the amino acid sequence of SEQ ID NO: 58.

4. The polypeptide of claim 3, wherein the PD-1 ligand is selected from PD-L1 and PD-L2.

5. The polypeptide of any one of claims 1 to 4, the variant ECD comprises an amino acid substitution at the serine residue at the position 87 (S87) with respect to SEQ ID NO: 57.

6. The polypeptide of any one of claims 1 to 5, the variant ECD comprises an amino acid substitution at the arginine residue at the position 86 (R86) with respect to SEQ ID NO: 57.

7. The polypeptide of any one of claims 1 to 6, the variant ECD comprises an amino acid substitution at the glutamine residue at the position 88 (Q88) with respect to SEQ ID NO: 57.

8. The polypeptide of any one of claims 5 to 7, wherein the S87 with respect to SEQ ID NO: 57 is replaced with an amino acid residue that is aromatic, aliphatic, hydrophobic, polar, hydrophilic, neutral of charge, negatively charged, positively charged or combination thereof.

9. The polypeptide of claim 8, wherein the S87 with respect to SEQ ID NO: 57 is replaced with an amino acid residue that is an hydrophilic, polar and positively charged residue is selected from lysine (K) and arginine (R).

10. The polypeptide of claim 8, wherein the S87 with respect to SEQ ID NO: 57 is replaced with an amino acid residue that is an aromatic, polar and positively charged hydrophilic residue, wherein the aromatic, polar and positively charged hydrophilic residue is histidine (H).

11. The polypeptide of claim 8, wherein the S87 with respect to SEQ ID NO: 57 is replaced with a hydrophilic is a polar and negatively charged hydrophilic amino acid residue selected from aspartate (D) and glutamate (E).

12. The polypeptide of claim 8, wherein the S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

13. The polypeptide of claim 8, wherein the S87 with respect to SEQ ID NO: 57 is replaced with an hydrophilic, polar and neutral of charge amino acid residue selected from asparagine (N), glutamine (Q), threonine (T), proline (P), and cysteine (C).

14. The polypeptide of claim 13, wherein the S87 with respect to SEQ ID NO: 57 is replaced with cysteine (C).

15. The polypeptide of claim 5, wherein the variant ECD comprises a S87C substitution with respect to SEQ ID NO: 57.

16. The polypeptide of any one of claims 6 to 14, wherein the R86 with respect to SEQ ID NO: 57 is replaced with an amino acid residue that is aromatic, aliphatic, hydrophobic, polar, hydrophilic, neutral of charge, negatively charged, positively charged or combination thereof.

17. The polypeptide of claim 16, wherein the R86 with respect to SEQ ID NO: 57 is replaced with an hydrophilic, polar and positively charged residue, which is lysine.

18. The polypeptide of claim 16, wherein the R86 with respect to SEQ ID NO: 57 is replaced with an aromatic, polar and positively charged hydrophilic residue, which is histidine (H).

19. The polypeptide of claim 16, wherein the R86 with respect to SEQ ID NO: 57 is replaced with an hydrophilic, polar and neutral of charge amino acid residue selected from asparagine (N), glutamine (Q), serine (S), threonine (T), proline (P), and cysteine (C).

20. The polypeptide of claim 16, wherein the R86 with respect to SEQ ID NO: 57 is replaced with an hydrophilic is a polar and negatively charged hydrophilic amino acid residue selected from aspartate (D) and glutamate (E).

21. The polypeptide of claim 16, wherein the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

22. The polypeptide of claim 21, wherein the hydrophobic, aliphatic amino acid residue is alanine (A).

23. The polypeptide of claim 6, or claim 15, wherein the variant ECD comprises a R86A substitution with respect to SEQ ID NO: 57.

24. The polypeptide of any one of claims 7 to 23, wherein the Q88 with respect to SEQ ID NO: 57 is replaced with an amino acid residue that is aromatic, aliphatic, hydrophobic, polar, hydrophilic, neutral of charge, negatively charged, positively charged or combination thereof.

25. The polypeptide of claim 24, wherein the Q88 with respect to SEQ ID NO: 57 is replaced with an hydrophilic, polar and positively charged residue selected from arginine and lysine.

26. The polypeptide of claim 24, wherein the Q88 with respect to SEQ ID NO: 57 is replaced with an aromatic, polar and positively charged hydrophilic residue, which is histidine (H).

27. The polypeptide of claim 24, wherein the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C).

28. The polypeptide of claim 24, wherein the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

29. The polypeptide of claim 24, wherein the Q88 with respect to SEQ ID NO: 57 is replaced with an hydrophilic is a polar and negatively charged hydrophilic amino acid residue selected from aspartate (D) and glutamate (E).

30. The polypeptide of claim 21, wherein the polar and negatively charged hydrophilic amino acid residue is glutamate (E).

31. The polypeptide of claim 7, claim 15, or claim 23, wherein the variant ECD comprises a Q88E substitution with respect to SEQ ID NO: 57.

32. The polypeptide of any one of claims 1 to 31, wherein the variant ECD comprises an amino acid sequence that is at least 70%, or 75%, or 80%, or 85%, or 90% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

33. The polypeptide of any one of claims 1 to 32, wherein the variant ECD comprises an amino acid sequence that is at least 95%, or 96%, or 97%, or 98%, or 99% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

34. The polypeptide of claim 32, wherein the variant ECD comprises an amino acid selected from SEQ ID NOs: 59-62.

35. The polypeptide of any one of claims 1 to 34, wherein polypeptide is a recombinant fusion protein.

36. A nucleic acid encoding the chimeric protein of any one of claims 1 to 35.

37. The nucleic acid of claim 36, wherein the nucleic acid is an mRNA.

38. The nucleic acid of claim 37, wherein the nucleic acid is a DNA.

39. An expression vector, comprising a nucleic acid encoding the chimeric protein of claim 36.

40. A host cell, comprising the expression vector of claim 39, mRNA of claim 37, or DNA of claim 38.

41. A pharmaceutical composition, comprising a therapeutically effective amount of the chimeric protein of any one of claims 1 to 35, or the nucleic acid of any one of claims 36 to 38, or the expression vector of claim 39 or the host cell of claim 40.

42. A method of treating cancer or an inflammatory disease, comprising administering an effective amount of a pharmaceutical composition of claim 41 to a subject in need thereof.

43. A method of modulating a patient's immune response, comprising administering an effective amount of a pharmaceutical composition of claim 41 to a subject in need thereof.

44. A chimeric protein comprising:
(a) a variant extracellular domain (ECD) of PD-1, wherein the variant ECD comprises one or more substitutions at one or more amino acid residues corresponding to one or more of the arginine residue at the position 86 (R86), the serine residue at the position 87 (S87), and glutamine residue at the position 88 (Q88) with respect to SEQ ID NO: 57; and
(b) a carrier protein,
wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

45. The chimeric protein of claim 44, wherein the carrier protein is selected from albumin, transferrin, an Fc, or elastin-like protein, or a variant thereof.

46. The chimeric protein of claim 45, wherein the Fc domain is selected from an IgG Fc domain, an IgA Fc domain, an IgM Fc domain, an IgE Fc domain and an IgD Fc domain.

47. The chimeric protein of claim 46, wherein the IgG Fc domain is selected from an IgG1 Fc domain, an IgG2 Fc domain, an IgG3 Fc domain, and an IgG4 Fc domain.

48. The chimeric protein of claim 47, wherein the Fc domain comprises hinge-CH2-CH3 Fc domain derived from IgG4.

49. The chimeric protein of claim 48, wherein the Fc domain the hinge-CH2-CH3 Fc domain is derived from human IgG4.

50. The chimeric protein of claim 49, wherein the Fc domain comprises an amino acid sequence that is at least 95% identical to the amino acid sequence of SEQ ID NO: 1, SEQ ID NO: 2, or SEQ ID NO: 3.

51. The chimeric protein of claim 47, wherein the Fc domain comprises hinge-CH2-CH3 Fc domain derived from IgG1.

52. The chimeric protein of claim 51, wherein the Fc domain the hinge-CH2-CH3 Fc domain is derived from human IgG1.

53. The chimeric protein of any one of claims 44 to 52, wherein the chimeric protein further comprises one or more joining linkers, such joining linkers independently selected from SEQ ID NOs: 4-50.

54. The chimeric protein of claim 45, wherein the albumin is human serum albumin.

55. The chimeric protein of any one of claims 44 to 54, wherein the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58.

56. The chimeric protein of any one of claims 44 to 55, wherein the variant ECD has an affinity to a PD-1 ligand that is equivalent to the affinity of the wild type PD-1 ECD having an amino acid sequence that is identical to the amino acid sequence of SEQ ID NO: 58.

57. The chimeric protein of claim 56, wherein the PD-1 ligand is selected from PD-L1 and PD-L2.

58. The chimeric protein of any one of claims 44 to 57, the variant ECD comprises an amino acid substitution at the serine residue at the position 87 (S87) with respect to SEQ ID NO: 57.

59. The chimeric protein of any one of claims 44 to 58, the variant ECD comprises an amino acid substitution at the arginine residue at the position 86 (R86) with respect to SEQ ID NO: 57.

60. The chimeric protein of any one of claims 44 to 59, the variant ECD comprises an amino acid substitution at the glutamine residue at the position 88 (Q88) with respect to SEQ ID NO: 57.

61. The chimeric protein of any one of claims 44 to 60, wherein S87 with respect to SEQ ID NO: 57 is replaced with an amino acid residue that is aromatic, aliphatic, hydrophobic, polar, hydrophilic, neutral of charge, negatively charged, positively charged or combination thereof.

62. The chimeric protein of claim 61, wherein S87 with respect to SEQ ID NO: 57 is replaced with an amino acid residue that is an hydrophilic, polar and positively charged residue is selected from lysine (K) and arginine (R).

63. The chimeric protein of claim 61, wherein S87 with respect to SEQ ID NO: 57 is replaced with an amino acid residue that is an aromatic, polar and positively charged hydrophilic residue, wherein the aromatic, polar and positively charged hydrophilic residue is histidine (H).

64. The chimeric protein of claim 61, wherein S87 with respect to SEQ ID NO: 57 is replaced with a hydrophilic is a polar and negatively charged hydrophilic amino acid residue selected from aspartate (D) and glutamate (E).

65. The chimeric protein of claim 61, wherein S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

66. The chimeric protein of claim 61, wherein S87 with respect to SEQ ID NO: 57 is replaced with an hydrophilic, polar and neutral of charge amino acid residue selected from asparagine (N), glutamine (Q), threonine (T), proline (P), and cysteine (C).

67. The chimeric protein of claim 66, wherein S87 with respect to SEQ ID NO: 57 is replaced with cysteine (C).

68. The chimeric protein of claim 60, wherein the variant ECD comprises a S87C substitution with respect to SEQ ID NO: 57.

69. The chimeric protein of any one of claims 60 to 68, wherein the R86 with respect to SEQ ID NO: 57 is replaced with an amino acid residue that is aromatic, aliphatic, hydrophobic, polar, hydrophilic, neutral of charge, negatively charged, positively charged or combination thereof.

70. The chimeric protein of claim 69, wherein the R86 with respect to SEQ ID NO: 57 is replaced with an hydrophilic, polar and positively charged residue, which is lysine.

71. The chimeric protein of claim 69, wherein the R86 with respect to SEQ ID NO: 57 is replaced with an aromatic, polar and positively charged hydrophilic residue, which is histidine (H).

72. The chimeric protein of claim 69, wherein the R86 with respect to SEQ ID NO: 57 is replaced with an hydrophilic, polar and neutral of charge amino acid residue selected from asparagine (N), glutamine (Q), serine (S), threonine (T), proline (P), and cysteine (C).

73. The chimeric protein of claim 69, wherein the R86 with respect to SEQ ID NO: 57 is replaced with an hydrophilic is a polar and negatively charged hydrophilic amino acid residue selected from aspartate (D) and glutamate (E).

74. The chimeric protein of claim 69, wherein the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

75. The chimeric protein of claim 74, wherein the hydrophobic, aliphatic amino acid residue is alanine (A).

76. The chimeric protein of claim 60, or claim 68, wherein the variant ECD comprises a R86A substitution with respect to SEQ ID NO: 57.

77. The chimeric protein of any one of claims 60 to 76, wherein the Q88 with respect to SEQ ID NO: 57 is replaced with an amino acid residue that is aromatic, aliphatic, hydrophobic, polar, hydrophilic, neutral of charge, negatively charged, positively charged or combination thereof.

78. The chimeric protein of claim 77, wherein the Q88 with respect to SEQ ID NO: 57 is replaced with an hydrophilic, polar and positively charged residue selected from arginine and lysine.

79. The chimeric protein of claim 77, wherein the Q88 with respect to SEQ ID NO: 57 is replaced with an aromatic, polar and positively charged hydrophilic residue, which is histidine (H).

80. The chimeric protein of claim 77, wherein the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C).

81. The chimeric protein of claim 77, wherein the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

82. The chimeric protein of claim 77, wherein the Q88 with respect to SEQ ID NO: 57 is replaced with an hydrophilic is a polar and negatively charged hydrophilic amino acid residue selected from aspartate (D) and glutamate (E).

83. The chimeric protein of claim 82, wherein the polar and negatively charged hydrophilic amino acid residue is glutamate (E).

84. The chimeric protein of claim 60, claim 68, or claim 76, wherein the variant ECD comprises a Q88E substitution with respect to SEQ ID NO: 57.

85. The chimeric protein of any one of claims 44 to 84, wherein the variant ECD comprises an amino acid sequence that is at least 95%, or 96%, or 97%, or 98%, or 99% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

86. The chimeric protein of claim 85, wherein the variant ECD comprises an amino acid selected from SEQ ID NOs: 59-62.

87. The chimeric protein of any one of claims 44 to 86, wherein the variant ECD comprises an amino acid sequence that is at least 70%, or 75%, or 80%, or 85%, or 90% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

88. The chimeric protein of any one of claims 44 to 87, wherein chimeric protein is a recombinant chimeric protein.

89. A nucleic acid encoding the chimeric protein of any one of claims 44 to 88.

90. The nucleic acid of claim 89, wherein the nucleic acid is an mRNA.

91. The nucleic acid of claim 90, wherein the nucleic acid is a DNA.

92. An expression vector, comprising a nucleic acid encoding the chimeric protein of claim 89.

93. A host cell, comprising the expression vector of claim 92, mRNA of claim 90, or DNA of claim 91.

94. A pharmaceutical composition, comprising a therapeutically effective amount of the chimeric protein of any one of claims 44 to 88, or the nucleic acid of any one of claims 89 to 91, or the expression vector of claim 92 or the host cell of claim 93.

95. A method of treating cancer or an inflammatory disease, comprising administering an effective amount of a pharmaceutical composition of claim 94 to a subject in need thereof.

96. A method of modulating a patient's immune response, comprising administering an effective amount of a pharmaceutical composition of claim 94 to a subject in need thereof.

97. A chimeric protein comprising (a) a first domain comprising a variant extracellular domain (ECD) of PD-1, wherein the variant ECD comprises one or more substitutions at one or more amino acid residues corresponding to one or more of R86, S87, and Q88 with respect to SEQ ID NO: 57, (b) a second domain comprising an extracellular domain of a transmembrane protein, and (c) a linker, wherein the variant ECD and/or the chimeric protein has less affinity to pembrolizumab and/or nivolumab compared to a wild type PD-1 ECD having an amino acid sequence that is identical to the amino acid sequence of SEQ ID NO: 58.

98. The chimeric protein of claim 97, wherein the variant ECD and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58.

99. The chimeric protein of claim 97 or claim 98, wherein the variant ECD has an affinity to a PD-1 ligand that is equivalent to the affinity of the wild type PD-1 ECD having an amino acid sequence that is identical to the amino acid sequence of SEQ ID NO: 58.

100. The chimeric protein of claim 99, wherein the PD-1 ligand is selected from PD-L1 and PD-L2.

101. The chimeric protein of any one of claims 97 to 100, the variant ECD comprises an amino acid substitution at the serine residue at the position 87 (S87) with respect to SEQ ID NO: 57.

102. The chimeric protein of any one of claims 97 to 101 the variant ECD comprises an amino acid substitution at the arginine residue at the position 86 (R86) with respect to SEQ ID NO: 57.

103. The chimeric protein of any one of claims 97 to 102, the variant ECD comprises an amino acid substitution at the glutamine residue at the position 88 (Q88) with respect to SEQ ID NO: 57.

104. The chimeric protein of claim 103, wherein S87 with respect to SEQ ID NO: 57 is replaced with an amino acid residue that is aromatic, aliphatic, hydrophobic, polar, hydrophilic, neutral of charge, negatively charged, positively charged or combination thereof.

105. The chimeric protein of claim 104, wherein S87 with respect to SEQ ID NO: 57 is replaced with an amino acid residue that is an hydrophilic, polar and positively charged residue is selected from lysine (K) and arginine (R).

106. The chimeric protein of claim 104, wherein S87 with respect to SEQ ID NO: 57 is replaced with an amino acid residue that is an aromatic, polar and positively charged hydrophilic residue, wherein the aromatic, polar and positively charged hydrophilic residue is histidine (H).

107. The chimeric protein of claim 104, wherein S87 with respect to SEQ ID NO: 57 is replaced with a hydrophilic is a polar and negatively charged hydrophilic amino acid residue selected from aspartate (D) and glutamate (E).

108. The chimeric protein of claim 104, wherein S87 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

109. The chimeric protein of claim 104, wherein S87 with respect to SEQ ID NO: 57 is replaced with an hydrophilic, polar and neutral of charge amino acid residue selected from asparagine (N), glutamine (Q), threonine (T), proline (P), and cysteine (C).

110. The chimeric protein of claim 109, wherein S87 with respect to SEQ ID NO: 57 is replaced with cysteine (C).

111. The chimeric protein of claim 103, wherein the variant ECD comprises a S87C substitution with respect to SEQ ID NO: 57.

112. The chimeric protein of any one of claims 103 to 111, wherein the R86 with respect to SEQ ID NO: 57 is replaced with an amino acid residue that is aromatic, aliphatic, hydrophobic, polar, hydrophilic, neutral of charge, negatively charged, positively charged or combination thereof.

113. The chimeric protein of claim 112, wherein the R86 with respect to SEQ ID NO: 57 is replaced with an hydrophilic, polar and positively charged residue, which is lysine.

114. The chimeric protein of claim 112, wherein the R86 with respect to SEQ ID NO: 57 is replaced with an aromatic, polar and positively charged hydrophilic residue, which is histidine (H).

115. The chimeric protein of claim 112, wherein the R86 with respect to SEQ ID NO: 57 is replaced with an hydrophilic, polar and neutral of charge amino acid residue selected from asparagine (N), glutamine (Q), serine (S), threonine (T), proline (P), and cysteine (C).

116. The chimeric protein of claim 112, wherein the R86 with respect to SEQ ID NO: 57 is replaced with an hydrophilic is a polar and negatively charged hydrophilic amino acid residue selected from aspartate (D) and glutamate (E).

117. The chimeric protein of claim 112, wherein the R86 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

118. The chimeric protein of claim 117, wherein the hydrophobic, aliphatic amino acid residue is alanine (A).

119. The chimeric protein of claim 103 or claim 111, wherein the variant ECD comprises a R86A substitution with respect to SEQ ID NO: 57.

120. The chimeric protein of any one of claims 103 to 119, wherein the Q88 with respect to SEQ ID NO: 57 is replaced with an amino acid residue that is aromatic, aliphatic, hydrophobic, polar, hydrophilic, neutral of charge, negatively charged, positively charged or combination thereof.

121. The chimeric protein of claim 120, wherein the Q88 with respect to SEQ ID NO: 57 is replaced with an hydrophilic, polar and positively charged residue selected from arginine and lysine.

122. The chimeric protein of claim 120, wherein the Q88 with respect to SEQ ID NO: 57 is replaced with an aromatic, polar and positively charged hydrophilic residue, which is histidine (H).

123. The chimeric protein of claim 120, wherein the Q88 with respect to SEQ ID NO: 57 is replaced with an hydrophilic, polar and neutral of charge amino acid residue selected from asparagine (N), glutamine (Q), serine (S), threonine (T), proline (P), and cysteine (C).

124. The chimeric protein of claim 120, wherein the Q88 with respect to SEQ ID NO: 57 is replaced with a hydrophobic, aliphatic amino acid residue is selected from glycine (G), alanine (A), leucine (L), isoleucine (I), methionine (M), and valine (V), or a hydrophobic, aromatic amino acid selected from phenylalanine (F), tryptophan (W), and tyrosine (Y).

125. The chimeric protein of claim 120, wherein the Q88 with respect to SEQ ID NO: 57 is replaced with an hydrophilic is a polar and negatively charged hydrophilic amino acid residue selected from aspartate (D) and glutamate (E).

126. The chimeric protein of claim 125, wherein the polar and negatively charged hydrophilic amino acid residue is glutamate (E).

127. The chimeric protein of claim 103, claim 111, or claim 119, wherein the variant ECD comprises a Q88E substitution with respect to SEQ ID NO: 57.

128. The chimeric protein of any one of claims 97 to 127, wherein the variant ECD comprises an amino acid sequence that is at least 70%, or 75%, or 80%, or 85%, or 90% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

129. The chimeric protein of any one of claims 97 to 128, wherein the variant ECD comprises an amino acid sequence that is at least 95%, or 96%, or 97%, or 98%, or 99% identical to the amino acid sequence selected from SEQ ID NOs: 58-62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

130. The chimeric protein of claim 129, wherein the variant ECD comprises an amino acid selected from SEQ ID NOs: 59-62.

131. The chimeric protein of any one of claims 97 to 130, wherein the linker comprises a polypeptide selected from a flexible amino acid sequence, an IgG hinge region, and an antibody sequence.

132. The chimeric protein of any one of claims 97 to 131, wherein the linker comprises at least one cysteine residue capable of forming a disulfide bond.

133. The chimeric protein of any one of claims 97 to 132, wherein the linker comprises an Fc domain.

134. The chimeric protein of claim 133, wherein the Fc domain is selected from an IgG Fc domain, an IgA Fc domain, an IgM Fc domain, an IgE Fc domain and an IgD Fc domain.

135. The chimeric protein of claim 134, wherein the IgG Fc domain is selected from an IgG1 Fc domain, an IgG2 Fc domain, an IgG3 Fc domain, and an IgG4 Fc domain.

136. The chimeric protein of claim 135, wherein the Fc domain comprises hinge-CH2-CH3 Fc domain derived from IgG4.

137. The chimeric protein of claim 136, wherein the Fc domain the hinge-CH2-CH3 Fc domain is derived from human IgG4.

138. The chimeric protein of claim 137, wherein the Fc domain comprises an amino acid sequence that is at least 95% identical to the amino acid sequence of SEQ ID NO: 1, SEQ ID NO: 2, or SEQ ID NO: 3.

139. The chimeric protein of claim 135, wherein the Fc domain comprises hinge-CH2-CH3 Fc domain derived from IgG1.

140. The chimeric protein of claim 139, wherein the Fc domain the hinge-CH2-CH3 Fc domain is derived from human IgG1.

141. The chimeric protein of any one of claims 97 to 140, wherein the chimeric protein further comprises one or more joining linkers, such joining linkers independently selected from SEQ ID NOs: 4-50.

142. The chimeric protein of claim 141, wherein the linker comprises two or more joining linkers each joining linker independently selected from SEQ ID NOs: 4-50; wherein one joining linker is N terminal to the hinge-CH2-CH3 Fc domain and another joining linker is C terminal to the hinge-CH2-CH3 Fc domain.

143. The chimeric protein of any one of claims 97 to 142, wherein the transmembrane protein is a Type II transmembrane protein.

144. The chimeric protein of claim 143, wherein the Type II transmembrane protein is selected from 4-1BBL, OX40L, CD70, CD30L, CD40L, GITRL, TL1A, and LIGHT.

145. The chimeric protein of claim 144, wherein the second domain is 4-1BBL, wherein the 4-1BBL is capable of binding to a 4-1BBL receptor.

146. The chimeric protein of claim 145, wherein the 4-1BBL receptor is 4-1BB.

147. The chimeric protein of claim 145 or claim 146, wherein the second domain comprises an amino acid sequence that is at least 95% identical to the amino acid sequence of SEQ ID NO: 64.

148. The chimeric protein of claim 144, wherein the second domain is OX40L, wherein the OX40L is capable of binding to an OX40L receptor.

149. The chimeric protein of claim 148, wherein the OX40L receptor is OX40.

150. The chimeric protein of claim 148 or claim 149, wherein the second domain comprises an amino acid sequence that is at least 95% identical to the amino acid sequence of SEQ ID NO: 65.

151. The chimeric protein of claim 144, wherein the second domain is CD70, wherein the CD70 is capable of binding to its ligand.

152. The chimeric protein of claim 151, wherein the ligand is CD27.

153. The chimeric protein of claim 151 or claim 152, wherein the second domain comprises an amino acid sequence that is at least 95% identical to the amino acid sequence of SEQ ID NO: 66.

154. The chimeric protein of claim 144, wherein the second domain is CD30L, wherein the CD30L is capable of binding to a CD30L receptor.

155. The chimeric protein of claim 154, wherein the CD30L receptor is CD30.

156. The chimeric protein of claim 154 or claim 155, wherein the second domain comprises an amino acid sequence that is at least 95% identical to the amino acid sequence of SEQ ID NO: 67.

157. The chimeric protein of claim 144, wherein the second domain is CD40L, wherein the CD40L is capable of binding to a CD40L receptor.

158. The chimeric protein of claim 157, wherein the CD40L receptor is CD40.

159. The chimeric protein of claim 157 or claim 158, wherein the second domain comprises an amino acid sequence that is at least 95% identical to the amino acid sequence of SEQ ID NO: 68.

160. The chimeric protein of claim 144, wherein the second domain is GITRL, wherein the GITRL is capable of binding to a GITRL receptor.

161. The chimeric protein of claim 160, wherein the GITRL receptor is GITR.

162. The chimeric protein of claim 160 or claim 161, wherein the second domain comprises an amino acid sequence that is at least 95% identical to the amino acid sequence of SEQ ID NO: 69.

163. The chimeric protein of claim 144, wherein the second domain is TL1A, wherein the TL1A is capable of binding to a TL1A ligand.

164. The chimeric protein of claim 163, wherein the TL1A ligand is DR3.

165. The chimeric protein of claim 163 or claim 164, wherein the second domain comprises an amino acid sequence that is at least 95% identical to the amino acid sequence of SEQ ID NO: 70.

166. The chimeric protein of claim 144, wherein the second domain is LIGHT, wherein the LIGHT is capable of binding to a LIGHT ligand.

167. The chimeric protein of claim 166, wherein the LIGHT ligand is TR2/TNFRSF14/HVEM.

168. The chimeric protein of claim 166 or claim 167, wherein the second domain comprises an amino acid sequence that is at least 95% identical to the amino acid sequence of SEQ ID NO: 71.

169. The chimeric protein of any one of claims 97 to 168, wherein chimeric protein is a recombinant chimeric protein.

170. A nucleic acid encoding the chimeric protein of any one of claims 97 to 169.

171. The nucleic acid of claim 170, wherein the nucleic acid is an mRNA.

172. The nucleic acid of claim 171, wherein the nucleic acid is a DNA.

173. An expression vector, comprising a nucleic acid encoding the chimeric protein of claim 170.

174. A host cell, comprising the expression vector of claim 173, mRNA of claim 171, or DNA of claim 172.

175. A pharmaceutical composition, comprising a therapeutically effective amount of the chimeric protein of any one of claims 97 to 169, or the nucleic acid of any one of claims 170 to 172, or the expression vector of claim 174 or the host cell of claim 174.

176. A method of treating cancer or an inflammatory disease, comprising administering an effective amount of a pharmaceutical composition of claim 175 to a subject in need thereof.

177. A method of modulating a patient's immune response, comprising administering an effective amount of a pharmaceutical composition of claim 175 to a subject in need thereof.

178. A recombinant protein comprising variant extracellular domain (ECD) of PD-1 comprising an amino acid sequence that is 95% identical to the amino acids of (i) SEQ ID NO: 59, or (ii) SEQ ID NO: 60, (iii) SEQ ID NO: 61, or (iv) SEQ ID NO: 62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57.

179. A chimeric protein comprising:
(a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57; and
(b) a carrier protein selected from selected from albumin, transferrin, an Fc, or elastin-like protein, or a variant thereof.

180. A chimeric protein comprising (a) a variant extracellular domain (ECD) of PD-1 comprising an amino acid sequence that is 95% identical to amino acids 24 to 178 of the amino acid sequence of (i) SEQ ID NO: 59, or (ii) SEQ ID NO: 60, (iii) SEQ ID NO: 61, or (iv) SEQ ID NO: 62, wherein the variant extracellular domain of PD-1 comprises:
an alanine residue at the position 86 corresponding to SEQ ID NO: 57;
a cysteine residue at the position 87 corresponding to SEQ ID NO: 57; and/or
a glutamic acid at the position 88 corresponding to SEQ ID NO: 57;
(b) a second domain comprising an extracellular domain of a Type II transmembrane protein selected from 4-1BBL, OX40L, CD70, CD30L, CD40L, GITRL, TL1A, and LIGHT, and (c) a linker.

181. The chimeric protein of claim 180, wherein the chimeric protein comprises a general structure of:
N terminus-(a)-(c)-(b)-C terminus,
wherein:
(c) is the linker, and
(b) is the second domain comprising an extracellular domain of Type II transmembrane protein.

182. The recombinant protein of claim 178, chimeric protein of claim 180, or the chimeric protein of claim 181, wherein the variant ECD, recombinant protein, the chimeric protein, and/or the chimeric protein has an affinity to pembrolizumab and/or nivolumab that is less by at least 3 fold, or at least 10 fold, or at least 30 fold, or at least 100 fold, or at least 300 fold, or at least 1000 fold compared to the wild type PD-1 ECD having an amino acid sequence that is identical to the amino acid sequence of SEQ ID NO: 58.

183. The recombinant protein of claim 178, chimeric protein of claim 180, or the chimeric protein of claim 181, wherein the variant ECD, recombinant protein, the chimeric protein, and/or the chimeric protein has an affinity to a PD-1 ligand that is equivalent to the affinity of the wild type PD-1 ECD having an amino acid sequence that is identical to the amino acid sequence of SEQ ID NO: 58.

184. The chimeric protein of claim 183, wherein the PD-1 ligand is selected from PD-L1 and PD-L2.

185. A nucleic acid encoding the recombinant protein of any one of claims 178, or 182 to 184, chimeric protein of any one of claims 180, or 182 to 184, or the chimeric protein of any one of claims 181 to 184.

186. The nucleic acid of claim 185, wherein the nucleic acid is an mRNA.

187. The nucleic acid of claim 186, wherein the nucleic acid is a DNA.

188. An expression vector, comprising a nucleic acid encoding the chimeric protein of claim 185.

189. A host cell, comprising the expression vector of claim 188, mRNA of claim 186, or DNA of claim 187.

190. A pharmaceutical composition, comprising a therapeutically effective amount of the recombinant protein of any one of claims 178, or 182 to 184, chimeric protein of any one of claims 180, or 182 to 184, the chimeric protein of any one of claims 181 to 184, or the nucleic acid of any one of claims 185 to 187, or the expression vector of claim 188 or the host cell of claim 189.

191. A method of treating cancer or an inflammatory disease, comprising administering an effective amount of a pharmaceutical composition of claim 190 to a subject in need thereof.

192. A method of modulating a patient's immune response, comprising administering an effective amount of a pharmaceutical composition of claim 190 to a subject in need thereof.
